(19) United States
(12) Patent Application Publication
(10) Pub. No.: US 2025/0136932 A1
(43) Pub. Date: May 1, 2025

(54) COMPOSITIONS AND METHODS FOR CULTURING AND MAINTAINING TROPHOBLAST STEM CELLS

(71) Applicant: NORTH CAROLINA STATE UNIVERSITY, Raleigh, NC (US)

(72) Inventors: Balaji Rao, Raleigh, NC (US); Victoria Karakis, Raleigh, NC (US)

(21) Appl. No.: 18/833,330

(22) PCT Filed: Jan. 27, 2023
(86) PCT No.: PCT/US2023/011678

§ 371 Date: Jul. 25, 2024

Related U.S. Application Data
(60) Provisional application No. 63/303,630, filed on Jan. 27, 2022.

(51) Int. Cl. C12N 5/073

(52) U.S. Cl. CPC C12N 5/0605; C12N 2500/36; C12N 2500/38; C12N 2501/065; C12N 2501/11; C12N 2501/119; C12N 2501/12; C12N 2501/16; C12N 2501/727

(57) ABSTRACT

The present disclosure provides compositions and methods related to the culturing of trophoblast stem cells (TSCs). In particular, the present disclosure provides novel formulations and methods for inducing and maintaining trophoblast stem cells obtained from cytotrophoblasts (CTBs) obtained from a placenta at birth. The compositions and methods described herein allow for the development of in vitro models of early human placental development and establish a platform for therapeutic discoveries.

### CROSS REFERENCE TO RELATED APPLICATION

This application claims priority to and the benefit of U.S. Provisional Patent Application No. 63/303,630 filed Jan. 27, 2022, which is incorporated herein by reference in its entirety and for all purposes.

### GOVERNMENT SUPPORT

This invention was made with government support under grant numbers HD093982 and HD106184 awarded by the National Institutes of Health. The government has certain rights in the invention.

### FIELD

The present disclosure provides compositions and methods related to the culturing of trophoblast stem cells (TSCs). In particular, the present disclosure provides novel formulations and methods for inducing or deriving, and maintaining trophoblast stem cells obtained from human pluripotent stem cells and placental cytotrophoblasts (CTBs), including CTBs obtained from a placenta at birth. The compositions and methods described herein allow for the development of in vitro models of early human placental development and establish a platform for therapeutic discoveries.

### BACKGROUND

Despite relevance to maternal and fetal health, molecular mechanisms underlying early trophoblast development are poorly understood. Upon embryo implantation, the outer trophectoderm layer of the blastocyst-stage embryo gives rise to the epithelial cytotrophoblast (CTB), which forms all trophoblast cell types of the placenta. The CTB differentiates into the multinucleate syncytiotrophoblast (STB) that overlays the CTB layer in the placental villi, and cells of the extravillous trophoblast (EVT) lineage. EVT differentiation of CTBs gives rise to proliferative column trophoblasts adjacent to the villus tip. Subsequently, column trophoblasts at the distal end undergo an epithelial-to-mesenchymal transition to form mature mesenchymal EVTs. The mature EVTs exhibit invasive behavior and play a critical role in remodeling uterine arteries and establishing adequate perfusion of maternal blood in the placenta. Abnormalities in trophoblast differentiation during early gestation are associated with placental pathologies and adverse outcomes in maternal and fetal health. However, despite its importance, molecular mechanisms regulating trophoblast differentiation remain poorly understood due to restrictions on research with human embryos, and limited availability of primary placental samples from early gestation for in vitro studies. Furthermore, significant differences between early placental development in humans and other animals limits the use of animal models.

Additionally, it has been long recognized that placental cells cross into maternal circulation during pregnancy. Studies in mice conducted in the context of heart failure showed that CDX2+ placental cells home into sites of injury and regenerate the heart. Importantly, these cells are immune privileged and avoid rejection by the immune system. Other studies in mice have shown that CDX2+ stem cells can be used to treat acute lung injury. Thus, CDX2+ stem can potentially revolutionize regenerative medicine. Specifically, they have broad differentiation potential, are safe, and do not need additional differentiation steps prior to use in therapy.

However, a major problem to date has been the inability to obtain CDX2+ stem cells from human placentas at birth. CDX2 is expressed very early in human development and obtaining these cells from human embryos or early termination of pregnancy for medicinal applications is impractical. In addition to ethical limitations, culture conditions did not exist for expanding these cells in culture. Thus, there is a need for compositions and methods for establishing in vitro culture systems for mechanistic studies on human trophoblast induction and maintenance.

### SUMMARY

Embodiments of the present disclosure include a chemically defined cell culture medium for inducing and maintaining human trophoblast stem cells (hTSCs) from cytotrophoblasts (CTBs). In accordance with these embodiments, the medium includes a GSK3β inhibitor, an activin/nodal inhibitor, and at least one growth factor.

In some embodiments, the CTBs are obtained from a human placenta at birth. In some embodiments, the CTBs are obtained during or after the second trimester of human pregnancy.

In some embodiments, the hTSCs exhibit altered expression of one or more of CDX2, TFAP2C, YAP, TEAD4, KRT7, p63, and GATA3. In some embodiments, the hTSCs express CDX2.

In some embodiments, the medium further comprises nicotinamide, nicotinamide riboside, nicotinamide mononucleotide or derivatives, variants, and salts thereof. In some embodiments, the nicotinamide is present in the medium at a concentration from about 1 mM to about 20 mM.

In some embodiments, the medium further comprises lactate, or derivatives, variants, and salts thereof. In some embodiments, the lactate is sodium lactate and it is present in the medium at a concentration from about 2 mM to about 20 mM.

In some embodiments, the at least one growth factor is fibroblast growth factor 10 (FGF10), hepatocyte growth factor (HGF), and/or epidermal growth factor (EGF), and any derivatives or variants thereof. In some embodiments, the at least one growth factor is present in the media at a concentration from about 1 ng/mL to about 100 ng/mL.

In some embodiments, the medium further comprises a sphingosine 1-phosphate receptor (S1PR) agonist. In some embodiments, the S1PR agonist is an agonist of S1PR1, S1PR2, or S1PR3. In some embodiments, the S1PR agonist is S1P. In some embodiments, the S1PR agonist is selected from the group consisting of CYM5442, CYM5541, CYM5520, A971432, Ceralifimod, CS2100, CYM50260, CYM50308, FTY720, GSK2018682, RP001, SEW2871, TC-G1006, TC-SP14, and any derivatives or variants thereof. In some embodiments, the S1PR agonist is an agonist of S1PR2. In some embodiments, the S1PR agonist is present in the medium at a concentration from about 1 μM to about 10 μM.

In some embodiments, the GSK3β inhibitor is CHIR99021 or any derivatives or variants thereof. In some embodiments, the GSK3β inhibitor is present in the medium at a concentration from about 0.5 μM to about 5 μM.

In some embodiments, the activin/nodal inhibitor is SB431542 or A83-01, and any derivatives or variants thereof. In some embodiments, the activin/nodal inhibitor is present in the medium at a concentration from about 0.2 μM to about 4 μM.

In some embodiments, the medium comprises ascorbic acid at a concentration from about 25 μg/mL to about 150 μg/mL.

In some embodiments, the medium further comprises lysophosphatidic acid (LPA), and/or an LPA receptor agonist. In some embodiments, the LPA receptor agonist comprises 2-[[3-(1,3-dioxo-1H-benz[de]isoquinolin-2(3H)-yl)propyl]thio]benzoic acid (GRI 977143) or 1-O-9Z-Octadecenoyl-sn-glyceryl-3-phosphoric acid (1-Oleoyl lysophosphatidic acid), or any salts thereof. In some embodiments, the LPA or LPA receptor agonist is present in the medium at a concentration from about 0.1 nM to about 5 μM.

In some embodiments, the medium further comprises decanoic acid and/or dimethyl alpha-ketoglutarate (DMKG). In some embodiments, the decanoic acid or the DMKG is present in the medium at a concentration from about 100 nM to about 10 mM.

In some embodiments, the medium comprises DMEM and/or F12 basal medium.

In some embodiments, the medium further comprises glucose at a concentration of 20 mM or less.

In some embodiments, the medium further comprises an inhibitor of mitochondrial pyruvate uptake. In some embodiments, the mitochondrial pyruvate uptake inhibitor comprises α-cyano-β-(1-phenylindol-3-yl)-acrylate (UK5099). In some embodiments, the mitochondrial pyruvate uptake inhibitor is present in the medium at a concentration ranging from about 1 nM to about 500 nM.

In some embodiments, the medium further comprises bovine serum albumin (BSA). In some embodiments, the BSA is a lipid-rich BSA composition. In some embodiments, the lipid-rich BSA composition comprises phospholipids and/or other hydrophobic lipids. In some embodiments, the phospholipids comprise sphingoine-1-phosphate (S1P) and/or lysophosphatidic acid (LPA). In some embodiments, the lipid-rich BSA composition is Albumax, and wherein the Albumax is present in the medium at a concentration ranging from 0.05% to about 1.0%.

In some embodiments, the medium comprises oxygen levels that are above levels that are considered to be low oxygen levels for the purposes of cell culture. In some embodiments, the medium comprises oxygen levels that are at least 1%. In some embodiments, the medium comprises oxygen levels that are at least 5%. In some embodiments, the medium comprises oxygen levels that are at least 10%. In some embodiments, the medium comprises oxygen levels that are at least 15%. In some embodiments, the medium comprises oxygen levels that are at least 20%. In some embodiments, the medium comprises oxygen levels that are considered to be normoxic levels for the purposes of cell culture (normoxia). In some embodiments, the medium comprises oxygen levels that are from about 20% to about 21%.

Embodiments of the present disclosure also include a chemically defined cell culture medium for inducing and maintaining human trophoblast stem cells (hTSCs) from cytotrophoblasts (CTBs). In accordance with these embodiments, the medium includes a GSK3β inhibitor, an activin/nodal inhibitor, at least one growth factor, a histone deacetylase (HDAC) inhibitor, and a Rho-associated, coiled-coil containing protein kinase (ROCK) inhibitor.

In some embodiments, the CTBs are obtained from a human placenta at birth. In some embodiments, the CTBs are obtained during or after the second trimester of human pregnancy.

In some embodiments, the hTSCs exhibit altered expression of one or more of CDX2, TFAP2C, YAP, TEAD4, KRT7, p63, and GATA3. In some embodiments, the hTSCs express CDX2.

In some embodiments, the at least one growth factor is epidermal growth factor (EGF).

In some embodiments, the GSK3B inhibitor is CHIR99021 or any derivatives or variants thereof.

In some embodiments, the activin/nodal inhibitor is SB431542 or A83-01, and any derivatives or variants thereof.

In some embodiments, the HDAC inhibitor is valproic acid (VPA).

In some embodiments, the ROCK inhibitor is Y27632.

In some embodiments, the medium further comprises an inhibitor of mitochondrial pyruvate uptake. In some embodiments, the mitochondrial pyruvate uptake inhibitor comprises α-cyano-β-(1-phenylindol-3-yl)-acrylate (UK5099). In some embodiments, the mitochondrial pyruvate uptake inhibitor is present in the medium at a concentration ranging from about 1 nM to about 500 nM.

In some embodiments, the medium further comprises bovine serum albumin (BSA). In some embodiments, the BSA is a lipid-rich BSA composition. In some embodiments, the lipid-rich BSA composition comprises phospholipids and/or other hydrophobic lipids. In some embodiments, the phospholipids comprise sphingoine-1-phosphate (S1P) and/or lysophosphatidic acid (LPA). In some embodiments, the lipid-rich BSA composition is Albumax, and wherein the Albumax is present in the medium at a concentration ranging from 0.05% to about 1.0%.

In some embodiments, the medium comprises oxygen levels that are above levels that are considered to be low oxygen levels for the purposes of cell culture. In some embodiments, the medium comprises oxygen levels that are at least 1%. In some embodiments, the medium comprises oxygen levels that are at least 5%. In some embodiments, the medium comprises oxygen levels that are at least 10%. In some embodiments, the medium comprises oxygen levels that are at least 15%. In some embodiments, the medium comprises oxygen levels that are at least 20%. In some embodiments, the medium comprises oxygen levels that are considered to be normoxic levels for the purposes of cell culture (normoxia). In some embodiments, the medium comprises oxygen levels that are from about 20% to about 21%.

Embodiments of the present disclosure also include a method for inducing and maintaining human trophoblast stem cells (hTSCs) from cytotrophoblasts (CTBs) ex vivo. In accordance with these embodiments, the method includes obtaining CTBs from placentas at birth, and culturing the CTBs in any medium described in the present disclosure for at least 2 passages. In some embodiments, the hTSCs express CDX2.

Embodiments of the present disclosure also include an ex vivo human trophoblast stem cell (hTSC) derived from a cytotrophoblast (CTB) obtained from a placenta at birth. In some embodiments, the hTSC expresses CDX2.

Embodiments of the present disclosure also include a container comprising an ex vivo human trophoblast stem cell (hTSC) derived from a cytotrophoblast (CTB) obtained from a placenta at birth. In some embodiments, the hTSC expresses CDX2. In some embodiments, the container comprises any medium described in the present disclosure.

### BRIEF DESCRIPTION OF THE DRAWINGS

FIGS. 1A-1B: Representative images of CDX2+ hTSCs obtained from CTBs at term demonstrating expression of various characteristic markers, including CDX2, YAP, TFAP2C, p63, TEAD4 and GATA3, when cultured in DTM7 medium (FIG. 1A) and DTME medium (FIG. 1B).

FIGS. 2A-2B: Similar marker expression between term cells with and without UK and AlbuMAX. Note that cells can be culture in media without UK and Albumax only if they are transitioned from medium containing UK and Albumax. (A) Confocal images of CTB21126R+ term hTSCs in TSCM with addition of UK5099 and AlbuMAX, staining for GATA3, AP-2γ, YAP, Oct4, KRT7, and TEAD4. Nuclei were stained with DAPI. (B) Confocal images of CTB21126R+ term hTSCs in TSCM, staining for GATA3, AP-2γ, YAP, Oct4, KRT7, and TEAD4. Nuclei were stained with DAPI. Scale bars are 100 μm for all images unless specified otherwise.

FIGS. 3A-3B: CTs in TSCM express CTB markers but not CDX2 (A) Confocal images of CT29 hTSCs in TSCM, staining for GATA3, TEAD4, p63, CDX2, HIF1α, HIF2α, KRT7, Oct4, AP-2γ, and YAP. Nuclei were stained with DAPI. (B) Confocal images of CT30 hTSCs in TSCM, staining for GATA3, TEAD4, p63, CDX2, HIF1α, HIF2α, KRT7, Oct4, AP-2γ, and YAP. Nuclei were stained with DAPI. Scale bars are 100 μm for all images unless specified otherwise.

FIGS. 4A-4D: hTSCs express p63 and CDX2 and do not express HIF1α or HIF2α. (A) Confocal images of CTB21126R+ term hTSCs in TSCM with addition of UK5099 and AlbuMAX, staining for CDX2, p63, HIF1α, and HIF2α. Nuclei were stained with DAPI. (B) Confocal images of CTB21126R+ term hTSCs in TSCM (transitioned from TSCM with the addition of UK5099 and Albumax), staining for CDX2, p63, HIF1α, HIF2α. Nuclei were stained with DAPI. (C) Quantitative analysis of p63 and CDX2 expression intensity in CTB21126R+ hTSCs. Positive control are SC102A-1 hTSCs on day 3 of trophoblast differentiation as previously described. Analysis was performed in MATLAB and at least 2 biological replicates were used. The white circle represents the mean and the black bar represents the median. (***p<0.0005). (D) Quantitative analysis of HIF1α and HIF2α expression intensity in CTB21126R+ hTSCs. Analysis was performed in MATLAB and at least 2 biological replicates were used. The white circle represents the mean and the black bar represents the median. Scale bars are 100 μm for all images unless specified otherwise.

FIGS. 5A-5F: Differentiation of term cells with and without UK and AlbuMAX. (A) Quantitative analysis of HLA-G expression intensity in day 6 EVTs from CT30 hTSCs cultured in TSCM and CTB21126R+ hTSCs cultured in TSCM or TSCM with the addition of UK5099 and AlbuMAX obtained by the method previously described. Analysis was performed in MATLAB and at least 2 biological replicates were used. The white circle represents the mean and the black bar represents the median. (***p<0.0005). (B) Quantitative analysis of Notch1 expression intensity in day 6 EVTs from CT30 hTSCs cultured in TSCM and CTB21126R+ hTSCs cultured in TSCM or TSCM with the addition of UK5099 and AlbuMAX obtained by the method previously described. Analysis was performed in MATLAB and at least 2 biological replicates were used. The white circle represents the mean and the black bar represents the median. (***p<0.0005). (C) Confocal images of EVTs from CTB21126R+ hTSCs cultured in TSCM or TSCM with the addition of UK5099 and AlbuMAX obtained by the method previously described, staining for HLA-G and Notch1. Nuclei were stained with DAPI. (D) Fluorescent image of STB from CTB21126R+ hTSCs cultured in TSCM or TSCM with the addition of UK5099 and AlbuMAX obtained by the method previously described. Nuclei were stained with DAPI. Membrane was stained with Di-8-ANEPPS cell membrane stain. Scale bar is 50 μm. (E) Fusion efficiency of STB from CTB21126R+ hTSCs derived by the methods as previously described compared to CTB21126R+ hTSCs cultured in TSCM or TSCM+UK+A (negative control). Fusion index is calculated as (N−S)/T where N is the number of nuclei in the syncytia, S is the number of syncytia, and T is the total number of nuclei counted. (***p<0.0005, ns, not significant. Error bars, S.D., n=3). (F) Confocal images of STB from CTB21126R+ hTSCs cultured in TSCM or TSCM with the addition of UK5099 and AlbuMAX obtained by the method previously described, staining for hCG and SDC-1. Nuclei were stained with DAPI. Scale bars are 100 μm for all images unless specified otherwise.

FIGS. 6A-6F: Differentiation of term cells with and without UK and AlbuMAX using Matrigel and forskolin; cells without UK and AlbuMAX were initially cultured in the presence of UK and AlbuMAX. (A) Quantitative analysis of HLA-G expression intensity in day 6 EVTs from CT30 hTSCs cultured in TSCM and CTB21126R+ hTSCs cultured in TSCM or TSCM with the addition of UK5099 and AlbuMAX obtained by the method described by Okae et al. Analysis was performed in MATLAB and at least 2 biological replicates were used. The white circle represents the mean and the black bar represents the median. (***p<0.0005). (B) Quantitative analysis of Notch1 expression intensity in day 6 EVTs from CT30 hTSCs cultured in TSCM and CTB21126R+ hTSCs cultured in TSCM or TSCM with the addition of UK5099 and AlbuMAX obtained by the method described by Okae et al. Analysis was performed in MATLAB and at least 2 biological replicates were used. The white circle represents the mean and the black bar represents the median. (***p<0.0005). (C) Confocal images of EVTs from CTB21126R+ hTSCs cultured in TSCM or TSCM with the addition of UK5099 and AlbuMAX obtained by the method described by Okae et al., staining for HLA-G and Notch1. Nuclei were stained with DAPI. (D) Fluorescent image of STB from CTB21126R+ hTSCs cultured in TSCM or TSCM with the addition of UK5099 and AlbuMAX obtained by the method described by Okae et al. Nuclei were stained with DAPI. Membrane was stained with Di-8-ANEPPS cell membrane stain. Scale bar is 50 μm. (E) Fusion efficiency of STB from CTB21126R+ hTSCs derived by the method described by Okae et al. compared to CTB21126R+ hTSCs cultured in TSCM or TSCM+UK+A (negative control). Fusion index is calculated as (N−S)/T where N is the number of nuclei in the syncytia, S is the number of syncytia, and T is the total number of nuclei counted. (***p<0.0005, ns, not significant. Error bars, S.D., n=3). (F) Confocal images of STB from CTB21126R+ hTSCs cultured in TSCM or TSCM with the addition of UK5099 and AlbuMAX obtained by the method described by Okae et al., staining for hCG and SDC-1. Nuclei were stained with DAPI. Scale bars are 100 μm for all images unless specified otherwise.

FIGS. 7A-7F: hTSCs express are not formed from TSCM without supplementation. (A) Confocal images of CTB21126R+ term hTSCs in TSCM, staining for GATA3, TEAD4, p63, CDX2, HIF1α, HIF2α, KRT7, OCT4, AP-2γ, and YAP. Nuclei were stained with DAPI. (B) Quantitative analysis of p63 and CDX2 expression intensity in CTB21126R+ hTSCs. Analysis was performed in MATLAB and at least 2 biological replicates were used. The white circle represents the mean and the black bar represents the median. (***p<0.0005). (C) Confocal images of EVTs and STB from CTB21126R+ hTSCs cultured in TSCM obtained by the method previously described, staining for HLA-G and Notch1 for EVTs and hCG and SDC-1 for STB. Nuclei were stained with DAPI. (D) Quantitative analysis of HLA-G and Notch1 expression intensity in EVTs from CTB21126R+ hTSCs obtained by the method previously described. Analysis was performed in MATLAB and at least 2 biological replicates were used. The white circle represents the mean and the black bar represents the median. (E) Confocal images of EVTs and STB from CTB21126R+ hTSCs cultured in TSCM obtained by the method described by Okae et al., staining for HLA-G and Notch 1 for EVTs and hCG and SDC-1 for STB. Nuclei were stained with DAPI. (F) Quantitative analysis of HLA-G and Notch1 expression intensity in EVTs from CTB21126R+ hTSCs by the method described by Okae et al. Analysis was performed in MATLAB and at least 2 biological replicates were used. The white circle represents the mean and the black bar represents the median. Scale bars are 100 μm for all images unless specified otherwise.

FIGS. 8A-8F: UK5099 is necessary for hTSC derivation from term CTB. (A) Confocal images of CTB21126R+ term hTSCs in TSCM+AlbuMAX, staining for GATA3, TEAD4, p63, CDX2, HIF1α, HIF2α, KRT7, OCT4, AP-2γ, and YAP. Nuclei were stained with DAPI. (B) Quantitative analysis of p63 and CDX2 expression intensity in CTB21126R+ hTSCs. Analysis was performed in MATLAB and at least 2 biological replicates were used. The white circle represents the mean and the black bar represents the median. (***p<0.0005). (C) Confocal images of EVTs and STB from CTB21126R+ hTSCs cultured in TSCM+AlbuMAX without the addition of UK5099 obtained by the method previously described, staining for HLA-G and Notch1 for EVTs and hCG and SDC-1 for STB. Nuclei were stained with DAPI. (D) Quantitative analysis of HLA-G and Notch1 expression intensity in EVTs from CTB21126R+ hTSCs obtained by the method previously described. Analysis was performed in MATLAB and at least 2 biological replicates were used. The white circle represents the mean and the black bar represents the median. (E) Confocal images of EVTs and STB from CTB21126R+ hTSCs cultured in TSCM+AlbuMAX without the addition of UK5099 obtained by the method described by Okae et al., staining for HLA-G and Notch1 for EVTs and hCG and SDC-1 for STB. Nuclei were stained with DAPI. (F) Quantitative analysis of HLA-G and Notch1 expression intensity in EVTs from CTB21126R+ hTSCs by the method described by Okae et al. Analysis was performed in MATLAB and at least 2 biological replicates were used. The white circle represents the mean and the black bar represents the median. Scale bars are 100 μm for all images unless specified otherwise.

FIGS. 9A-9F: hTSCs express are not formed from TSCM+UK+LPA. (A) Confocal images of CTB21126R+ term hTSCs in TSCM+UK+LPA, staining for GATA3, TEAD4, p63, CDX2, HIF1α, HIF2α, KRT7, OCT4, AP-2γ, and YAP. Nuclei were stained with DAPI. (B) Quantitative analysis of p63 and CDX2 expression intensity in CTB21126R+ hTSCs. Analysis was performed in MATLAB and at least 2 biological replicates were used. The white circle represents the mean and the black bar represents the median. (***p<0.0005). (C) Confocal images of EVTs and STB from CTB21126R+ hTSCs cultured in TSCM+UK+LPA obtained by the method previously described, staining for HLA-G and Notch1 for EVTs and hCG and SDC-1 for STB. Nuclei were stained with DAPI. (D) Quantitative analysis of HLA-G and Notch1 expression intensity in EVTs from CTB21126R+ hTSCs obtained by the method previously described. Analysis was performed in MATLAB and at least 2 biological replicates were used. The white circle represents the mean and the black bar represents the median. (E) Confocal images of EVTs and STB from CTB21126R+ hTSCs cultured in TSCM+UK+LPA obtained by the method described by Okae et al., staining for HLA-G and Notch1 for EVTs and hCG and SDC-1 for STB. Nuclei were stained with DAPI. (F) Quantitative analysis of HLA-G and Notch1 expression intensity in EVTs from CTB21126R+ hTSCs by the method described by Okae et al. Analysis was performed in MATLAB and at least 2 biological replicates were used. The white circle represents the mean and the black bar represents the median. Scale bars are 100 μm for all images unless specified otherwise.

### DETAILED DESCRIPTION

Trophoblasts are the principal cell types in the placenta. Embodiments of the present disclosure describe a method and cell culture medium formulation for culture of human trophoblast stem cells (hTSCs). hTSCs cultured in this medium exhibit expression of the protein marker CDX2, which is associated with stem cells of the trophectoderm layer of the blastocyst stage embryo. Significantly, using this medium it is possible to derive hTSCs from villous cytotrophoblast cells of the placenta at birth, which is an abundantly available resource. It is important to note that derivation of hTSCs from term placentas (at birth) has not been reported to date. hTSCs have only been derived from first trimester placental samples (obtained during elective termination of pregnancy) or from human pluripotent stem cells. In fact, it has been argued that such stem cells exist only in the first trimester placenta.

Despite the vital role of the placenta in maternal and fetal health, the molecular mechanisms underlying human placental development remain poorly understood, due to restrictions on research with human embryos and fetal tissues, and their limited availability. Moreover, there are significant differences between placental development in humans and commonly used animal models. Embodiments of the present disclosure allow for development of in vitro models of early human placental development. This in turn will allow for applications such as drug/toxicant testing. Additionally, there is evidence in the mouse that stem cells of the placenta have value in regenerative medicine. However, to date such stem cells have been unable to be isolated/maintained from human placentas at birth. Thus, embodiments of the present disclosure will enable the exploration and potential application of human placental stem cells in regenerative medicine applications.

By days 3 to 4 after fertilization, the first cell lineage decision occurs, the segregation between the inner cell mass (ICM), an inner compact cluster of cells and precursor to the human embryo, and an outer layer of cells known as the trophectoderm (TE) and the precursor to the human placenta. Together, they make up what is called the human blastocyst which implants into the maternal uterus. Immediately following implantation, TE cells differentiate to form the syncytiotrophoblast (STB), a multinucleate cell that forms a mantle over the blastocyst and is the only cell in contact with the maternal cells. By the second week, projections of proliferating cells break through the mantle in rows to form primary villi. The cells develop into what's known as the villous cytotrophoblast (CTs), which continue to fuse with the existing STB layer and proliferate and further protrude through the syncytium to form the expansive network of placental villi. These villi can either remain free floating within the intervillous space or protrude further and become anchored to the maternal decidua, securing the placenta to the uterus.

For floating villi to anchor, CTs proliferate and again break through the syncytiotrophoblast layer and expand laterally to form the trophoblastic shell which is composed of cells that differentiate towards the extravillous trophoblast lineage (EVTs)². CTs first form proximal column cytotrophoblasts (CCTs) that proliferate rapidly near the anchored villi. Distal CCTs on the outer edge of the shell cease mitosis and dissociate from the proliferate columns to become migratory or invasive. Invasive EVTs invade into the decidua and the inner third of the myometrium whereas migratory EVTs migrate down the lumen to remodel the maternal uterine spiral arteries.

Some of the key transcription factors that regulate blastocyst formation are CDX2 and OCT4. At the 8-cell stage before compaction, OCT4 is first detected. After blastocyst formation however, on day 5, both CDX2 and OCT4 can be seen in the TE but on the 6th day, OCT4 expression is restricted to the ICM whereas CDX2 is restricted to the TE. Importantly, CDX2 expression is dependent on the initial presence of OCT4 since CRISPR-driven knockout of OCT4 in developing human embryos significantly reduced CDX2 expression in the TE. Yet, cells of the ICM gained CDX2 expression in OCT4-KO embryos, indicating OCT4 may repress CDX2 expression. This is starkly different from the CTs in the later stage villous placenta. On days 12-14 when the primary villi begin to form, they gain expression of a key CT marker, p63. They also retain expression of CDX2 at first before expression depletes significantly beginning mid-first trimester. Other factors that help characterize CTs are GATA3, TCF1, YAP, and AP-2γ.

Recently, a side population of CTs have been identified that compose of about 3.5% of the cytotrophoblast population on average throughout gestation. These cells are TEAD4+, ELF5+ and CDX2+ but lack the β4 subunit of the main population of villous trophoblasts. It is believed that this subpopulation of cells is the main stem cell compartment with the ability to differentiation down both syncytiotrophoblast and extravillous trophoblast pathways. Fibroblast growth factor 4 (FGF4) can induce ELF5 expression through FGF receptor 2 (FGFR2) in mouse trophoblasts and ELF5+ cells are mostly FGFR2+ as well. FGF4 expression likely originates from maternal endometrial glands. Importantly, CDX2 and ELF5 levels deplete significantly after the mid-first trimester and are mostly present only in the side-population stem cell compartment. Indeed, the ELF5 promoter is mostly hypomethylated in villous cytotrophoblasts, allowing for expression and a positive feedback loop with CDX2 and EOMES. Importantly, TEAD4 inhibits expression of key STB markers GCM1, OVOLI, hCG, and ENDOU⁹. GCM1 in turn activates syncytin 1 and 2 to drive cell fusion in STB.

For CTs to differentiate down the EVT lineage, cells first gain expression of Notch1 which inhibits expression of key CT markers TEAD4 and p63. It is believed that HIF drives CCT differentiation since culture in low oxygen directs CTB towards EVT differentiation and that HIF complexes were required for this differentiation. Additionally, hypoxia induces expression of ASCL2 and Notch1 in proximal column cytotrophoblasts. Proximal CCTs also express VE-Cadherin and MYC. Distal EVTs gain expression of HLA-G, TCF-4, Notch2, and ErbB2³. Mature EVTs that have broken off from the CCTs retain expression of HLA-G and lose expression of Notch1.

Most of the knowledge of mechanisms and transcription factors that regulate placental formation stems from in vivo studies with the mouse model. However, there are extreme differences that exist between the mouse and the human pregnancy. Mainly because these substantial differentiation events all commence before the third week of pregnancy, the very early stages of placental development remain poorly understood. Therefore, more recently, the field has begun focusing on using human in vitro models to simulate early placental development. Using hiPSCs or fibroblasts, researchers have been able to develop in vitro “blastoids” that are similar to the day 5 human post-implantation blastocyst. Additionally, primary human trophoblast stem cells (hTSCs) derived from first trimester placentas or blastocysts can be cultured in vitro and differentiate to EVT and STB.

These models, however, are unable to explain pregnancy disorders, which typically occur later in the pregnancy. Therefore, there has been a significant push to develop models to form hTSCs from term trophoblast cells. Term placenta is much more abundant than first trimester placentas, and it is possible to confirm healthy versus disease placentas and develop more relevant placental disease models. Recently, hTSCs have been derived from term cytotrophoblasts under hypoxic conditions but was not achieved in normoxia. In the present disclosure, the derivation of hTSCs from term cells at normoxia by supplementation with a pyruvate uptake inhibitor, UK5099 and AlbuMAX is described. Additionally, after a few passages these cells can be transferred to the same media as hTSCs derived from first trimester samples and have a similar RNA profile and can differentiate in the same manner as the first trimester hTSCs.

The recent advancement of the human trophoblast stem cell in vitro model has led to significant advancements in the understanding of trophoblast biology. However, human trophoblast stem cells cultured in TSCM alone failed to be derived from term placenta and could only be derived from 6-8 week placenta samples or blastocysts at normoxia. More recently, however, culturing trophoblast cells from term placenta at 1% oxygen in TSCM led to the derivation of hTSCs. In the present disclosure, it was demonstrated that trophoblast stem cells are derived from term placentas using TSCM supplemented with UK5099 and AlbuMAX under normoxic culture conditions. These hTSCs express key CTB markers GATA3, KRT7, AP-2γ, YAP, TEAD4, and p63. These hTSCs were also able to be transferred to TSCM from TSCM+UK5099+AlbuMAX and maintained expression of these relevant trophoblast markers. hTSCs cultured in both TSCM+UK5099+AlbuMAX and those transferred to TSCM showed the ability to differentiate into EVTs and STB using both the 1-step differentiation protocol and Okae's 2-step differentiation protocol. EVTs expressed key EVT markers HLA-G and Notch1 and STB expressed key STB markers hCG and SDC-1. These results together demonstrate the ability to successfully derive and culture hTSCs derived from term cytotrophoblasts.

Interestingly, hTSCs derived from term cytotrophoblasts express CDX2, whereas hTSCs derived from first trimester trophoblasts do not. CDX2 expression is important because it is characteristic of the villous cytotrophoblast stem cell compartment, a side population of cells that are TEAD4+, ELF5+, and CDX2+ but lack the β4 subunit of the main population of villous trophoblasts. Cells of this stem cell compartment have the ability to differentiate down both the STB and the EVT lineages. Importantly, however, CDX2 expression was seen even in conditions that did not permit differentiation to these lineages which points to differences between the various media compositions causing the ability to differentiate and is not simply a result of CDX2 expression. Interestingly, CDX2 expression has also observed in hPSC-derived trophoblast cells, albeit these cells were not cultured in TSCM. Also, when UK5099 and AlbuMAX were dropped from the media, CDX2 levels decreased. However, CDX2 expression was still seen in term-derived hTSCs cultured in TSCM alone and term-derived hTSCs still had higher CDX2 expression than hTSCs cultured from first-trimester placentas. Furthermore, cells directly thawed in TSCM have higher CDX2 expression compared to both TSCM+UK5099+AlbuMAX and cells passage into TSCM from TSCM+UK5099+AlbuMAX. On the other hand, cells cultured in TSCM+Albumax without the addition of UK5099 had lower CDX2 expression compared to TSCM+UK5099+AlbuMAX. Neither condition had the ability to differentiate to EVT or STB therefore, changes in CDX2 expression intensities do not seem to correlate with the ability to differentiate to EVT or STB effectively.

Several conditions that were explored for culturing hTSCs from term cytotrophoblasts could support cell growth and proliferation. However, not all of these cells had the ability to differentiate into both EVT and STB lineages. Cells thawed directly into TSCM, cells cultured in TSCM+AlbuMAX without the addition of UK5099, and cells cultured in TSCM+UK5099+LPA all could not differentiate into EVT and STB lineages. It was previously found that hPSC-derived hTSCs cultured in a 4-component defined medium led to the formation of CDX2+ cells with a similar marker expression and transcriptome profile as cells of the trophectoderm. Interestingly, these cells expressed CDX2 and could differentiate down the STB lineage but not down the EVT lineage. However, as described further herein, cells cultured in conditions that did not lead to differentiation still retained CDX2 expression, indicating that the inability to differentiate is not correlated with CDX2 expression. Importantly, it has been previously shown that HIF1α and HIF2α expression was seen in EVT but not STB differentiation. The inability to upregulate HIF signaling in EVT differentiation could explain impaired differentiation towards EVT, but this explanation does not explain the inability for these cells to form STB. This, along with differences in the hTSC conditions, led to the idea that cells cultured in these conditions are not true trophoblast stem cells. Importantly, AP-2g was all but absent from TSCM+UK+LPA and TSCM+AlbuMAX without UK5099. P63 was also non-nuclear in TSCM+UK+LPA and when hTSCs were thawed directly into TSCM. It is importantly to note that differences were seen between cells thawed directly into TSCM and cells thawed into TSCM+UK+AlbuMAX and transferred to TSCM after 5 passaged. This means that the reprogramming of term trophoblast cells towards hTSCs occur within the first 5 passages, making this culture condition extremely important in the derivation. After time, however, the culture can be manipulated and altered, after hTSCs are already established.

Thanks to the ease of access for term placentas compared to first trimester placentas and blastocysts, hTSC lines can easily be created from many term placentas with a wide variety of genetic backgrounds. This will help elucidate further mechanisms involved in placental development. Additionally, the ability to derive and stably culture term trophoblast stem cells expand the ability to study different placental pathologies, as most diseases do not present themselves within the first few weeks of pregnancy. hTSC disease models from various placental disorders can be created and compared to one another to better understand placental disease. Finally, these cells cultured in TSCM+UK5099+AlbuMAX demonstrates both expression of important markers for trophoblast stem cells along with the stem cell marker, CDX2.

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

As used herein, the term “animal” refers to any animal (e.g., a mammal), including, but not limited to, humans, non-human primates, pigs, rodents (e.g., mice, rats, etc.), flies, and the like.

As used herein, the term “subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, etc.) and a human). In some embodiments, the subject may be a human or a non-human. In one embodiment, the subject is a human. The subject or patient may be undergoing various forms of treatment.

The term “cell culture process” generally refers to the process by which cells are grown or maintained under controlled conditions. The cell culture process may take place in vitro or ex vivo. In some embodiments, a cell culture process has both an expansion phase and a production phase. In some embodiments, the expansion and production phases are separated by a transition or shift phase. “Culturing” a cell refers to contacting a cell with a cell culture medium under conditions suitable to for growing or maintaining the cell. A “cell culture” can also refer to a solution containing cells.

The terms “medium” and “cell culture medium” (plural, “media”) generally refer to a nutrient source used for growing or maintaining cells. As is understood by a person of ordinary skill in the art, the nutrient source may contain components required by the cell for growth and/or survival or may contain components that aid in cell growth and/or survival. Vitamins, essential or non-essential amino acids (e.g., cysteine and cystine), and trace elements (e.g., copper) are examples of medium components. A cell culture medium may also be supplemented (e.g., with a “medium supplement” or “supplement” with any one or more of a component that aids the cell culture process.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. COMPOSITIONS AND METHODS

Human trophoblast stem cells (hTSCs) derived from blastocyst-stage embryos and first trimester placentas have emerged as a potentially powerful in vitro model for studies on early placental development. Recent studies have shown that hTSCs equivalent to those found in vivo can also be derived from human pluripotent stem cells. As described further herein, human trophoblast stem cells (hTSCs) have been derived from cytotrophoblasts of the term placenta. Notably, hTSCs cannot be derived from term placentas using the widely used culture conditions reported by Okae et al. (Okae, et al., Derivation of Human Trophoblast Stem Cells. Cell stem cell 2018, 22 (1), 50-63.e6. (doi.org/10.1016/j.stem.2017.11.004)). Okae et al. speculated that these cells are lost during or after the second trimester of pregnancy; however, results of the present disclosure demonstrate that this is not the case.

Derivation of hTSCs from term placentas will have two significant applications: (1) enable efficient generation of hTSCs from genetically diverse backgrounds, including from pathological pregnancies, and facilitate development of in vitro systems for drug evaluation/toxicity testing; and (2) hTSCs will likely have application in regenerative medicine. Placental cells are abundantly available, non-controversial, and studies in mouse suggest that they may be useful for treating conditions such as myocardial infarction and acute lung injury.

In accordance with these embodiments, the cell culture medium formulations provided herein generally include chemically defined medium, or growth medium suitable for the in vitro cell culture of human or animal cells. As would be recognized by one of ordinary skill in the art, serum-free media and chemically defined media are distinct. For example, serum-free media may contain undefined animal-derived products such as serum (purified from blood), hydrolysates, growth factors, hormones, carrier proteins, and attachment factors. These undefined animal-derived products will contain complex contaminants, such as the lipid content of albumin. In contrast, chemically defined media requires that the components be identified and have their exact concentrations known and is generally free of animal-derived components (e.g., fetal bovine serum, bovine serum, human serum).

Embodiments of the present disclosure include a chemically defined cell culture medium for inducing and maintaining human trophoblast stem cells (hTSCs) from cytotrophoblasts (CTBs). In accordance with these embodiments, the medium includes a GSK3B inhibitor, an activin/nodal inhibitor, and at least one growth factor. In some embodiments, the CTBs are obtained from a placenta at birth. That is, the CTBs are obtained from placentas after the birth of a child or at some point during or after the second trimester of human pregnancy.

In some embodiments, the hTSCs cultured in the chemically defined cell culture medium of the present disclosure can exhibit altered expression of one or more of CDX2, TFAP2C, YAP, TEAD4, KRT7, p63, and GATA3. In some embodiments, the hTSCs express CDX2, or any combinations thereof. Altered expression can include decreases or increases in expression of these genes/proteins, which reflect, or are characteristic of, hTSCs. Other phenotypic characteristics of hTSCs can also be assessed.

In some embodiments, the chemically defined cell culture medium of the present disclosure further comprises nicotinamide, nicotinamide riboside, nicotinamide mononucleotide or derivatives, variants, and salts thereof. In some embodiments, the nicotinamide is present in the medium at a concentration from about 1 mM to about 20 mM. In some embodiments, the nicotinamide is present in the medium at a concentration from about 1 mM to about 15 mM. In some embodiments, the nicotinamide is present in the medium at a concentration from about 1 mM to about 10 mM. In some embodiments, the nicotinamide is present in the medium at a concentration from about 1 mM to about 5 mM. In some embodiments, the nicotinamide is present in the medium at a concentration from about 5 mM to about 20 mM. In some embodiments, the nicotinamide is present in the medium at a concentration from about 10 mM to about 20 mM. In some embodiments, the nicotinamide is present in the medium at a concentration from about 15 mM to about 20 mM. In some embodiments, the nicotinamide is present in the medium at a concentration from about 5 mM to about 15 mM. In some embodiments, the nicotinamide is present in the medium at a concentration from about 10 mM to about 15 mM. In some embodiments, the nicotinamide is present in the medium at a concentration from about 5 mM to about 10 mM.

In some embodiments, the chemically defined cell culture medium of the present disclosure further comprises lactate, or derivatives, variants, and salts thereof. In some embodiments, the lactate is sodium lactate and it is present in the medium at a concentration from about 2 mM to about 20 mM. In some embodiments, the sodium lactate is present in the medium at a concentration from about 5 mM to about 20 mM. In some embodiments, the sodium lactate is present in the medium at a concentration from about 10 mM to about 20 mM. In some embodiments, the sodium lactate is present in the medium at a concentration from about 15 mM to about 20 mM. In some embodiments, the sodium lactate is present in the medium at a concentration from about 2 mM to about 15 mM. In some embodiments, the sodium lactate is present in the medium at a concentration from about 2 mM to about 10 mM. In some embodiments, the sodium lactate is present in the medium at a concentration from about 2 mM to about 5 mM. In some embodiments, the sodium lactate is present in the medium at a concentration from about 5 mM to about 20 mM. In some embodiments, the sodium lactate is present in the medium at a concentration from about 10 mM to about 20 mM. In some embodiments, the sodium lactate is present in the medium at a concentration from about 15 mM to about 20 mM. In some embodiments, the sodium lactate is present in the medium at a concentration from about 5 mM to about 15 mM. In some embodiments, the sodium lactate is present in the medium at a concentration from about 10 mM to about 15 mM. In some embodiments, the sodium lactate is present in the medium at a concentration from about 5 mM to about 10 mM.

In some embodiments, the at least one growth factor is fibroblast growth factor 10 (FGF10), hepatocyte growth factor (HGF), and/or epidermal growth factor (EGF), and any derivatives or variants thereof. In some embodiments, the at least one growth factor is present in the media at a concentration from about 1 ng/mL to about 100 ng/mL. In some embodiments, the at least one growth factor is present in the media at a concentration from about 1 ng/mL to about 75 ng/mL. In some embodiments, the at least one growth factor is present in the media at a concentration from about 1 ng/mL to about 50 ng/mL. In some embodiments, the at least one growth factor is present in the media at a concentration from about 1 ng/mL to about 25 ng/mL. In some embodiments, the at least one growth factor is present in the media at a concentration from about 10 ng/mL to about 100 ng/mL. In some embodiments, the at least one growth factor is present in the media at a concentration from about 25 ng/mL to about 100 ng/mL. In some embodiments, the at least one growth factor is present in the media at a concentration from about 50 ng/mL to about 100 ng/mL. In some embodiments, the at least one growth factor is present in the media at a concentration from about 75 ng/mL to about 100 ng/mL. In some embodiments, the at least one growth factor is present in the media at a concentration from about 25 ng/mL to about 75 ng/mL. In some embodiments, the at least one growth factor is present in the media at a concentration from about 50 ng/mL to about 100 ng/mL. In some embodiments, the at least one growth factor is present in the media at a concentration from about 50 ng/mL to about 75 ng/mL.

In some embodiments, the chemically defined cell culture medium of the present disclosure comprises a sphingosine 1-phosphate receptor (S1PR) agonist. In some embodiments the S1PR agonist is an agonist of S1PR1, S1PR2, or S1PR3. In some embodiments, the S1PR agonist is selected from the group consisting of CYM5442, CYM5541, CYM5520, A971432, Ceralifimod, CS2100, CYM50260, CYM50308, FTY720, GSK2018682, RP001, SEW2871, TC-G1006, TC-SP14, and any derivatives or variants thereof. In some embodiments, the S1PR agonist is S1P that is naturally derived or synthetic. In some embodiments, the S1PR agonist is an agonist of S1PR2.

In some embodiments, the S1PR agonist is present in the medium at a concentration from about 1 μM to about 10 μM. In some embodiments, the S1PR agonist is present in the medium at a concentration from about 1 μM to about 8 μM. In some embodiments, the S1PR agonist is present in the medium at a concentration from about 1 μM to about 6 μM. In some embodiments, the S1PR agonist is present in the medium at a concentration from about 1 μM to about 5 μM. In some embodiments, the S1PR agonist is present in the medium at a concentration from about 1 μM to about 4 μM. In some embodiments, the S1PR agonist is present in the medium at a concentration from about 1 μM to about 3 μM. In some embodiments, the S1PR agonist is present in the medium at a concentration from about 2 μM to about 10 μM. In some embodiments, the S1PR agonist is present in the medium at a concentration from about 2 μM to about 10 μM. In some embodiments, the S1PR agonist is present in the medium at a concentration from about 3 μM to about 10 μM. In some embodiments, the S1PR agonist is present in the medium at a concentration from about 4 μM to about 10 μM. In some embodiments, the S1PR agonist is present in the medium at a concentration from about 5 μM to about 10 μM. In some embodiments, the S1PR agonist is present in the medium at a concentration from about 6 μM to about 10 μM. In some embodiments, the S1PR agonist is present in the medium at a concentration from about 8 μM to about 10 μM. In some embodiments, the S1PR agonist is present in the medium at a concentration from about 2 μM to about 8 μM. In some embodiments, the S1PR agonist is present in the medium at a concentration from about 4 μM to about 6 μM.

In some embodiments, the chemically defined cell culture medium of the present disclosure comprises a GSK3β inhibitor. In some embodiments, the GSK3β inhibitor is CHIR99021 or any derivatives or variants thereof. In some embodiments, the GSK3β inhibitor is present in the medium at a concentration from about 0.5 μM to about 5 μM. In some embodiments, the GSK3β inhibitor is present in the medium at a concentration from about 1 μM to about 5 μM. In some embodiments, the GSK3β inhibitor is present in the medium at a concentration from about 2 μM to about 5 μM. In some embodiments, the GSK3β inhibitor is present in the medium at a concentration from about 3 μM to about 5 μM. In some embodiments, the GSK3β inhibitor is present in the medium at a concentration from about 4 μM to about 5 μM. In some embodiments, the GSK3β inhibitor is present in the medium at a concentration from about 0.5 μM to about 4 μM. In some embodiments, the GSK3β inhibitor is present in the medium at a concentration from about 0.5 μM to about 3 μM. In some embodiments, the GSK3β inhibitor is present in the medium at a concentration from about 0.5 μM to about 2 μM. In some embodiments, the GSK3β inhibitor is present in the medium at a concentration from about 0.5 μM to about 1 μM. In some embodiments, the GSK3β inhibitor is present in the medium at a concentration from about 1 μM to about 4 μM. In some embodiments, the GSK3β inhibitor is present in the medium at a concentration from about 2 μM to about 3 μM.

In some embodiments, the chemically defined cell culture medium of the present disclosure comprises an activin/nodal inhibitor. In some embodiments, the activin/nodal inhibitor is SB431542 or A83-01, and any derivatives or variants thereof. In some embodiments, the activin/nodal inhibitor is present in the medium at a concentration from about 0.2 μM to about 4 μM. In some embodiments, the activin/nodal inhibitor is present in the medium at a concentration from about 0.2 μM to about 3 μM. In some embodiments, the activin/nodal inhibitor is present in the medium at a concentration from about 0.2 μM to about 2 μM. In some embodiments, the activin/nodal inhibitor is present in the medium at a concentration from about 0.2 μM to about 1 μM. In some embodiments, the activin/nodal inhibitor is present in the medium at a concentration from about 0.5 μM to about 4 μM. In some embodiments, the activin/nodal inhibitor is present in the medium at a concentration from about 1 μM to about 4 μM. In some embodiments, the activin/nodal inhibitor is present in the medium at a concentration from about 2 μM to about 4 μM. In some embodiments, the activin/nodal inhibitor is present in the medium at a concentration from about 3 μM to about 4 μM. In some embodiments, the activin/nodal inhibitor is present in the medium at a concentration from about 1 μM to about 3 μM.

In some embodiments, the chemically defined cell culture medium of the present disclosure comprises ascorbic acid. In some embodiments, the medium comprises ascorbic acid at a concentration from about 25 μg/mL to about 150 μg/mL. In some embodiments, the medium comprises ascorbic acid at a concentration from about 50 μg/mL to about 150 μg/mL. In some embodiments, the medium comprises ascorbic acid at a concentration from about 100 μg/mL to about 150 μg/mL. In some embodiments, the medium comprises ascorbic acid at a concentration from about 125 μg/mL to about 150 μg/mL. In some embodiments, the medium comprises ascorbic acid at a concentration from about 25 μg/mL to about 125 g/mL. In some embodiments, the medium comprises ascorbic acid at a concentration from about 25 g/mL to about 100 μg/mL. In some embodiments, the medium comprises ascorbic acid at a concentration from about 25 μg/mL to about 75 μg/mL. In some embodiments, the medium comprises ascorbic acid at a concentration from about 25 μg/mL to about 50 μg/mL. In some embodiments, the medium comprises ascorbic acid at a concentration from about 50 μg/mL to about 125 μg/mL. In some embodiments, the medium comprises ascorbic acid at a concentration from about 75 g/mL to about 100 μg/mL.

In some embodiments, the chemically defined cell culture medium of the present disclosure comprises lysophosphatidic acid (LPA), and/or an LPA receptor agonist. In some embodiments, the LPA receptor agonist comprises 2-[[3-(1,3-dioxo-1H-benz[de]isoquinolin-2(3H)-yl)propyl]thio]benzoic acid (GRI 977143) or 1-O-9Z-Octadecenoyl-sn-glyceryl-3-phosphoric acid (1-Oleoyl lysophosphatidic acid), or any salts thereof. In some embodiments, the LPA or LPA receptor agonist is present in the medium at a concentration from about 0.1 nM to about 5 M. In some embodiments, the LPA or LPA receptor agonist is present in the medium at a concentration from about 10 nM to about 5 μM. In some embodiments, the LPA or LPA receptor agonist is present in the medium at a concentration from about 100 nM to about 5 μM. In some embodiments, the LPA or LPA receptor agonist is present in the medium at a concentration from about 500 nM to about 5 μM. In some embodiments, the LPA or LPA receptor agonist is present in the medium at a concentration from about 1 μM to about 5 μM. In some embodiments, the LPA or LPA receptor agonist is present in the medium at a concentration from about 0.1 nM to about 1 M. In some embodiments, the LPA or LPA receptor agonist is present in the medium at a concentration from about 0.1 nM to about 500 nM. In some embodiments, the LPA or LPA receptor agonist is present in the medium at a concentration from about 0.1 nM to about 100 nM. In some embodiments, the LPA or LPA receptor agonist is present in the medium at a concentration from about 0.1 nM to about 10 nM. In some embodiments, the LPA or LPA receptor agonist is present in the medium at a concentration from about 100 nM to about 1 μM. In some embodiments, the LPA or LPA receptor agonist is present in the medium at a concentration from about 500 nM to about 1 μM.

In some embodiments, the chemically defined cell culture medium of the present disclosure comprises decanoic acid and/or dimethyl alpha-ketoglutarate (DMKG). In some embodiments, the medium comprises decanoic acid at a concentration ranging from about 100 nM to about 1 μM. In some embodiments, the medium comprises decanoic acid at a concentration ranging from about 250 nM to about 1 μM. In some embodiments, the medium comprises decanoic acid at a concentration ranging from about 500 nM to about 1 μM. In some embodiments, the medium comprises decanoic acid at a concentration ranging from about 750 nM to about 1 μM. In some embodiments, the medium comprises decanoic acid at a concentration ranging from about 100 nM to about 750 nM. In some embodiments, the medium comprises decanoic acid at a concentration ranging from about 100 nM to about 500 nM. In some embodiments, the medium comprises decanoic acid at a concentration ranging from about 100 nM to about 250 nM. In some embodiments, the medium comprises decanoic acid at a concentration ranging from about 250 nM to about 750 nM. In some embodiments, the medium comprises decanoic acid at a concentration ranging from about 250 nM to about 500 nM.

In some embodiments, the chemically defined cell culture medium of the present disclosure comprises decanoic acid and/or dimethyl alpha-ketoglutarate (DMKG). In some embodiments, the medium comprises DMKG at a concentration ranging from about 100 nM to about 10 mM. In some embodiments, the medium comprises DMKG at a concentration ranging from about 500 nM to about 10 mM. In some embodiments, the medium comprises DMKG at a concentration ranging from about 1 μM to about 10 mM. In some embodiments, the medium comprises DMKG at a concentration ranging from about 500 μM to about 10 mM. In some embodiments, the medium comprises DMKG at a concentration ranging from about 1 mM to about 10 mM. In some embodiments, the medium comprises DMKG at a concentration ranging from about 100 nM to about 1 mM. In some embodiments, the medium comprises DMKG at a concentration ranging from about 100 nM to about 500 μM. In some embodiments, the medium comprises DMKG at a concentration ranging from about 100 nM to about 1 μM. In some embodiments, the medium comprises DMKG at a concentration ranging from about 100 nM to about 500 nM. In some embodiments, the medium comprises DMKG at a concentration ranging from about 500 nM to about 1 μM. In some embodiments, the medium comprises DMKG at a concentration ranging from about 1 μM to about 1 mM.

In some embodiments, the chemically defined cell culture medium of the present disclosure comprises DMEM and/or F12 basal medium. In some embodiments, the chemically defined cell culture medium of the present disclosure comprises DMEM basal medium. In some embodiments, the chemically defined cell culture medium of the present disclosure comprises F12 basal medium. In some embodiments, the chemically defined cell culture medium of the present disclosure comprises DMEM and F12 basal medium.

In some embodiments, the chemically defined cell culture medium of the present disclosure further comprises glucose. In some embodiments, the medium includes glucose at a concentration of 20 mM or less. In some embodiments, the medium includes glucose at a concentration of 19 mM or less. In some embodiments, the medium includes glucose at a concentration of 18 mM or less. In some embodiments, the medium includes glucose at a concentration of 17 mM or less. In some embodiments, the medium includes glucose at a concentration of 16 mM or less. In some embodiments, the medium includes glucose at a concentration of 15 mM or less. In some embodiments, the medium includes glucose at a concentration of 14 mM or less. In some embodiments, the medium includes glucose at a concentration of 13 mM or less. In some embodiments, the medium includes glucose at a concentration of 12 mM or less. In some embodiments, the medium includes glucose at a concentration of 11 mM or less. In some embodiments, the medium includes glucose at a concentration of 10 mM or less. In some embodiments, the medium includes glucose at a concentration of 9 mM or less. In some embodiments, the medium includes glucose at a concentration of 8 mM or less. In some embodiments, the medium includes glucose at a concentration of 7 mM or less. In some embodiments, the medium includes glucose at a concentration of 6 mM or less. In some embodiments, the medium includes glucose at a concentration of 5 mM or less. In some embodiments, the medium includes glucose at a concentration of 4 mM or less. In some embodiments, the medium includes glucose at a concentration of 3 mM or less. In some embodiments, the medium includes glucose at a concentration of 2 mM or less. In some embodiments, the medium includes glucose at a concentration of 1 mM or less.

In some embodiments, the medium includes glucose at a concentration ranging from about 1 mM to about 20 mM. In some embodiments, the medium includes glucose at a concentration ranging from about 1 mM to about 18 mM. In some embodiments, the medium includes glucose at a concentration ranging from about 1 mM to about 16 mM. In some embodiments, the medium includes glucose at a concentration ranging from about 1 mM to about 14 mM. In some embodiments, the medium includes glucose at a concentration ranging from about 1 mM to about 12 mM. In some embodiments, the medium includes glucose at a concentration ranging from about 1 mM to about 10 mM. In some embodiments, the medium includes glucose at a concentration ranging from about 1 mM to about 9 mM. In some embodiments, the medium includes glucose at a concentration ranging from about 1 mM to about 8 mM. In some embodiments, the medium includes glucose at a concentration ranging from about 1 mM to about 7 mM. In some embodiments, the medium includes glucose at a concentration ranging from about 1 mM to about 6 mM. In some embodiments, the medium includes glucose at a concentration ranging from about 1 mM to about 5 mM. In some embodiments, the medium includes glucose at a concentration ranging from about 2 mM to about 20 mM. In some embodiments, the medium includes glucose at a concentration ranging from about 3 mM to about 15 mM. In some embodiments, the medium includes glucose at a concentration ranging from about 4 mM to about 10 mM. In some embodiments, the medium includes glucose at a concentration ranging from about 5 mM to about 20 mM. In some embodiments, the medium includes glucose at a concentration ranging from about 5 mM to about 15 mM. In some embodiments, the medium includes glucose at a concentration ranging from about 5 mM to about 10 mM. In some embodiments, the medium includes glucose at a concentration ranging from about 2 mM to about 8 mM. In some embodiments, the medium includes glucose at a concentration ranging from about 3 mM to about 6 mM. In some embodiments, the medium includes glucose at a concentration ranging from about 4 mM to about 5 mM.

In some embodiments, the chemically defined cell culture medium of the present disclosure comprises an inhibitor of mitochondrial pyruvate uptake. In some embodiments, the mitochondrial pyruvate uptake inhibitor comprises α-cyano-β-(1-phenylindol-3-yl)-acrylate (UK5099). In some embodiments, the mitochondrial pyruvate uptake inhibitor is present in the medium at a concentration ranging from about 1 nM to about 500 nM. In some embodiments, the mitochondrial pyruvate uptake inhibitor is present in the medium at a concentration ranging from about 25 nM to about 500 nM. In some embodiments, the mitochondrial pyruvate uptake inhibitor is present in the medium at a concentration ranging from about 50 nM to about 500 nM. In some embodiments, the mitochondrial pyruvate uptake inhibitor is present in the medium at a concentration ranging from about 100 nM to about 500 nM. In some embodiments, the mitochondrial pyruvate uptake inhibitor is present in the medium at a concentration ranging from about 200 nM to about 500 nM. In some embodiments, the mitochondrial pyruvate uptake inhibitor is present in the medium at a concentration ranging from about 300 nM to about 500 nM. In some embodiments, the mitochondrial pyruvate uptake inhibitor is present in the medium at a concentration ranging from about 400 nM to about 500 nM. In some embodiments, the mitochondrial pyruvate uptake inhibitor is present in the medium at a concentration ranging from about 1 nM to about 400 nM. In some embodiments, the mitochondrial pyruvate uptake inhibitor is present in the medium at a concentration ranging from about 1 nM to about 300 nM. In some embodiments, the mitochondrial pyruvate uptake inhibitor is present in the medium at a concentration ranging from about 1 nM to about 200 nM. In some embodiments, the mitochondrial pyruvate uptake inhibitor is present in the medium at a concentration ranging from about 1 nM to about 100 nM. In some embodiments, the mitochondrial pyruvate uptake inhibitor is present in the medium at a concentration ranging from about 1 nM to about 50 nM. In some embodiments, the mitochondrial pyruvate uptake inhibitor is present in the medium at a concentration ranging from about 1 nM to about 25 nM. In some embodiments, the mitochondrial pyruvate uptake inhibitor is present in the medium at a concentration ranging from about 10 nM to about 100 nM. In some embodiments, the mitochondrial pyruvate uptake inhibitor is present in the medium at a concentration ranging from about 25 nM to about 75 nM. In some embodiments, the mitochondrial pyruvate uptake inhibitor is present in the medium at a concentration ranging from about 50 nM to about 100 nM.

In some embodiments, the chemically defined cell culture medium of the present disclosure comprises bovine serum albumin (BSA). In some embodiments, the BSA is a lipid-rich BSA composition. In some embodiments, the lipid-rich BSA composition comprises phospholipids and/or other hydrophobic lipids. In some embodiments, the phospholipids comprise sphingoine-1-phosphate (S1P) and/or lysophosphatidic acid (LPA). In some embodiments, the lipid-rich BSA composition is Albumax, and wherein the Albumax is present in the medium at a concentration ranging from 0.05% to about 1.0%. In some embodiments, the lipid-rich BSA composition is Albumax, and wherein the Albumax is present in the medium at a concentration ranging from 0.1% to about 1.0%. In some embodiments, the lipid-rich BSA composition is Albumax, and wherein the Albumax is present in the medium at a concentration ranging from 0.25% to about 1.0%. In some embodiments, the lipid-rich BSA composition is Albumax, and wherein the Albumax is present in the medium at a concentration ranging from 0.5% to about 1.0%. In some embodiments, the lipid-rich BSA composition is Albumax, and wherein the Albumax is present in the medium at a concentration ranging from 0.75% to about 1.0%. In some embodiments, the lipid-rich BSA composition is Albumax, and wherein the Albumax is present in the medium at a concentration ranging from 0.05% to about 0.75%. In some embodiments, the lipid-rich BSA composition is Albumax, and wherein the Albumax is present in the medium at a concentration ranging from 0.05% to about 0.5%. In some embodiments, the lipid-rich BSA composition is Albumax, and wherein the Albumax is present in the medium at a concentration ranging from 0.05% to about 0.25%. In some embodiments, the lipid-rich BSA composition is Albumax, and wherein the Albumax is present in the medium at a concentration ranging from 0.05% to about 0.1%. In some embodiments, the lipid-rich BSA composition is Albumax, and wherein the Albumax is present in the medium at a concentration ranging from 0.05% to about 0.5%. In some embodiments, the lipid-rich BSA composition is Albumax, and wherein the Albumax is present in the medium at a concentration ranging from 0.1% to about 0.5%. In some embodiments, the lipid-rich BSA composition is Albumax, and wherein the Albumax is present in the medium at a concentration ranging from 0.25% to about 0.5%.

In some embodiments, the medium comprises oxygen levels that are above levels that are considered to be low oxygen levels for the purposes of cell culture. In some embodiments, the medium comprises oxygen levels that are at least 1%. In some embodiments, the medium comprises oxygen levels that are at least 5%. In some embodiments, the medium comprises oxygen levels that are at least 10%. In some embodiments, the medium comprises oxygen levels that are at least 15%. In some embodiments, the medium comprises oxygen levels that are at least 20%. In some embodiments, the medium comprises oxygen levels that are considered to be normoxic levels for the purposes of cell culture (normoxia). In some embodiments, the medium comprises oxygen levels that are from about 20% to about 21%.

In addition to the media formulations described above, and in accordance with the results and data of the present disclosure described below, embodiments provided herein also include a chemically defined cell culture medium for inducing and maintaining human trophoblast stem cells (hTSCs) from cytotrophoblasts (CTBs) that includes a GSK3β inhibitor, an activin/nodal inhibitor, at least one growth factor, a histone deacetylase (HDAC) inhibitor, and a Rho-associated, coiled-coil containing protein kinase (ROCK) inhibitor. In some embodiments, the CTBs are obtained from a placenta at birth. That is, the CTBs are obtained from placentas after the birth of a child or at some point during or after the second trimester of human pregnancy.

In some embodiments, the hTSCs cultured in the chemically defined cell culture medium of the present disclosure can exhibit altered expression of one or more of CDX2, TFAP2C, YAP, TEAD4, KRT7, p63, and GATA3. In some embodiments, the hTSCs express CDX2, or any combinations thereof. Altered expression can include decreases or increases in expression of these genes/proteins, which reflect, or are characteristic of, hTSCs. Other phenotypic characteristics of hTSCs can also be assessed.

In some embodiments, the at least one growth factor is epidermal growth factor (EGF). In some embodiments, the at least one growth factor is present in the media at a concentration from about 1 ng/mL to about 100 ng/mL. In some embodiments, the at least one growth factor is present in the media at a concentration from about 1 ng/mL to about 75 ng/mL. In some embodiments, the at least one growth factor is present in the media at a concentration from about 1 ng/mL to about 50 ng/mL. In some embodiments, the at least one growth factor is present in the media at a concentration from about 1 ng/mL to about 25 ng/mL. In some embodiments, the at least one growth factor is present in the media at a concentration from about 10 ng/mL to about 100 ng/mL. In some embodiments, the at least one growth factor is present in the media at a concentration from about 25 ng/mL to about 100 ng/mL. In some embodiments, the at least one growth factor is present in the media at a concentration from about 50 ng/mL to about 100 ng/mL. In some embodiments, the at least one growth factor is present in the media at a concentration from about 75 ng/mL to about 100 ng/mL. In some embodiments, the at least one growth factor is present in the media at a concentration from about 25 ng/mL to about 75 ng/mL. In some embodiments, the at least one growth factor is present in the media at a concentration from about 50 ng/mL to about 100 ng/mL. In some embodiments, the at least one growth factor is present in the media at a concentration from about 50 ng/mL to about 75 ng/mL.

In some embodiments, the chemically defined cell culture medium of the present disclosure comprises a GSK3β inhibitor. In some embodiments, the GSK3β inhibitor is CHIR99021 or any derivatives or variants thereof. In some embodiments, the GSK3β inhibitor is present in the medium at a concentration from about 0.5 μM to about 5 μM. In some embodiments, the GSK3β inhibitor is present in the medium at a concentration from about 1 μM to about 5 μM. In some embodiments, the GSK3β inhibitor is present in the medium at a concentration from about 2 μM to about 5 μM. In some embodiments, the GSK3β inhibitor is present in the medium at a concentration from about 3 μM to about 5 μM. In some embodiments, the GSK3β inhibitor is present in the medium at a concentration from about 4 μM to about 5 μM. In some embodiments, the GSK3β inhibitor is present in the medium at a concentration from about 0.5 μM to about 4 μM. In some embodiments, the GSK3β inhibitor is present in the medium at a concentration from about 0.5 μM to about 3 μM. In some embodiments, the GSK3β inhibitor is present in the medium at a concentration from about 0.5 μM to about 2 μM. In some embodiments, the GSK3β inhibitor is present in the medium at a concentration from about 0.5 μM to about 1 μM. In some embodiments, the GSK3β inhibitor is present in the medium at a concentration from about 1 μM to about 4 μM. In some embodiments, the GSK3β inhibitor is present in the medium at a concentration from about 2 μM to about 3 μM.

In some embodiments, the chemically defined cell culture medium of the present disclosure comprises an activin/nodal inhibitor. In some embodiments, the activin/nodal inhibitor is SB431542 or A83-01, and any derivatives or variants thereof. In some embodiments, the activin/nodal inhibitor is present in the medium at a concentration from about 0.2 M to about 4 μM. In some embodiments, the activin/nodal inhibitor is present in the medium at a concentration from about 0.2 μM to about 3 μM. In some embodiments, the activin/nodal inhibitor is present in the medium at a concentration from about 0.2 μM to about 2 μM. In some embodiments, the activin/nodal inhibitor is present in the medium at a concentration from about 0.2 μM to about 1 μM. In some embodiments, the activin/nodal inhibitor is present in the medium at a concentration from about 0.5 μM to about 4 μM. In some embodiments, the activin/nodal inhibitor is present in the medium at a concentration from about 1 μM to about 4 μM. In some embodiments, the activin/nodal inhibitor is present in the medium at a concentration from about 2 μM to about 4 μM. In some embodiments, the activin/nodal inhibitor is present in the medium at a concentration from about 3 μM to about 4 μM. In some embodiments, the activin/nodal inhibitor is present in the medium at a concentration from about 1 μM to about 3 μM.

In some embodiments, the chemically defined cell culture medium of the present disclosure comprises a histone deacetylase (HDAC) inhibitor. In some embodiments, the HDAC inhibitor is valproic acid (VPA). In some embodiments, the HDAC inhibitor is present in the medium at a concentration from about 0.1 mM to about 2.0 mM. In some embodiments, the HDAC inhibitor is present in the medium at a concentration from about 0.5 mM to about 2.0 mM. In some embodiments, the HDAC inhibitor is present in the medium at a concentration from about 1.5 mM to about 2.0 mM. In some embodiments, the HDAC inhibitor is present in the medium at a concentration from about 0.1 mM to about 1.5 mM. In some embodiments, the HDAC inhibitor is present in the medium at a concentration from about 0.1 mM to about 1.0 mM. In some embodiments, the HDAC inhibitor is present in the medium at a concentration from about 0.1 mM to about 0.5 mM. In some embodiments, the HDAC inhibitor is present in the medium at a concentration from about 0.4 mM to about 1.2 mM. In some embodiments, the HDAC inhibitor is present in the medium at a concentration from about 0.6 mM to about 1.0 mM.

In some embodiments, the chemically defined cell culture medium of the present disclosure comprises a Rho-associated, coiled-coil containing protein kinase (ROCK) inhibitor. In some embodiments, the ROCK inhibitor is Y27632. In some embodiments, the ROCK inhibitor is present in the medium at a concentration from about 1 μM to about 20 μM. In some embodiments, the ROCK inhibitor is present in the medium at a concentration from about 2 μM to about 20 μM. In some embodiments, the ROCK inhibitor is present in the medium at a concentration from about 5 μM to about 20 μM. In some embodiments, the ROCK inhibitor is present in the medium at a concentration from about 10 μM to about 20 μM. In some embodiments, the ROCK inhibitor is present in the medium at a concentration from about 15 μM to about 20 μM. In some embodiments, the ROCK inhibitor is present in the medium at a concentration from about 1 μM to about 15 μM. In some embodiments, the ROCK inhibitor is present in the medium at a concentration from about 1 μM to about 10 μM. In some embodiments, the ROCK inhibitor is present in the medium at a concentration from about 1 μM to about 5 μM. In some embodiments, the ROCK inhibitor is present in the medium at a concentration from about 2 μM to about 10 μM. In some embodiments, the ROCK inhibitor is present in the medium at a concentration from about 3 μM to about 7 μM.

In some embodiments, the chemically defined cell culture medium of the present disclosure comprises an inhibitor of mitochondrial pyruvate uptake. In some embodiments, the mitochondrial pyruvate uptake inhibitor comprises α-cyano-β-(1-phenylindol-3-yl)-acrylate (UK5099). In some embodiments, the mitochondrial pyruvate uptake inhibitor is present in the medium at a concentration ranging from about 1 nM to about 500 nM. In some embodiments, the mitochondrial pyruvate uptake inhibitor is present in the medium at a concentration ranging from about 25 nM to about 500 nM. In some embodiments, the mitochondrial pyruvate uptake inhibitor is present in the medium at a concentration ranging from about 50 nM to about 500 nM. In some embodiments, the mitochondrial pyruvate uptake inhibitor is present in the medium at a concentration ranging from about 100 nM to about 500 nM. In some embodiments, the mitochondrial pyruvate uptake inhibitor is present in the medium at a concentration ranging from about 200 nM to about 500 nM. In some embodiments, the mitochondrial pyruvate uptake inhibitor is present in the medium at a concentration ranging from about 300 nM to about 500 nM. In some embodiments, the mitochondrial pyruvate uptake inhibitor is present in the medium at a concentration ranging from about 400 nM to about 500 nM. In some embodiments, the mitochondrial pyruvate uptake inhibitor is present in the medium at a concentration ranging from about 1 nM to about 400 nM. In some embodiments, the mitochondrial pyruvate uptake inhibitor is present in the medium at a concentration ranging from about 1 nM to about 300 nM. In some embodiments, the mitochondrial pyruvate uptake inhibitor is present in the medium at a concentration ranging from about 1 nM to about 200 nM. In some embodiments, the mitochondrial pyruvate uptake inhibitor is present in the medium at a concentration ranging from about 1 nM to about 100 nM. In some embodiments, the mitochondrial pyruvate uptake inhibitor is present in the medium at a concentration ranging from about 1 nM to about 50 nM. In some embodiments, the mitochondrial pyruvate uptake inhibitor is present in the medium at a concentration ranging from about 1 nM to about 25 nM. In some embodiments, the mitochondrial pyruvate uptake inhibitor is present in the medium at a concentration ranging from about 10 nM to about 100 nM. In some embodiments, the mitochondrial pyruvate uptake inhibitor is present in the medium at a concentration ranging from about 25 nM to about 75 nM. In some embodiments, the mitochondrial pyruvate uptake inhibitor is present in the medium at a concentration ranging from about 50 nM to about 100 nM.

In some embodiments, the chemically defined cell culture medium of the present disclosure comprises bovine serum albumin (BSA). In some embodiments, the BSA is a lipid-rich BSA composition. In some embodiments, the lipid-rich BSA composition comprises phospholipids and/or other hydrophobic lipids. In some embodiments, the phospholipids comprise sphingoine-1-phosphate (S1P) and/or lysophosphatidic acid (LPA). In some embodiments, the lipid-rich BSA composition is Albumax, and wherein the Albumax is present in the medium at a concentration ranging from 0.05% to about 1.0%. In some embodiments, the lipid-rich BSA composition is Albumax, and wherein the Albumax is present in the medium at a concentration ranging from 0.1% to about 1.0%. In some embodiments, the lipid-rich BSA composition is Albumax, and wherein the Albumax is present in the medium at a concentration ranging from 0.25% to about 1.0%. In some embodiments, the lipid-rich BSA composition is Albumax, and wherein the Albumax is present in the medium at a concentration ranging from 0.5% to about 1.0%. In some embodiments, the lipid-rich BSA composition is Albumax, and wherein the Albumax is present in the medium at a concentration ranging from 0.75% to about 1.0%. In some embodiments, the lipid-rich BSA composition is Albumax, and wherein the Albumax is present in the medium at a concentration ranging from 0.05% to about 0.75%. In some embodiments, the lipid-rich BSA composition is Albumax, and wherein the Albumax is present in the medium at a concentration ranging from 0.05% to about 0.5%. In some embodiments, the lipid-rich BSA composition is Albumax, and wherein the Albumax is present in the medium at a concentration ranging from 0.05% to about 0.25%. In some embodiments, the lipid-rich BSA composition is Albumax, and wherein the Albumax is present in the medium at a concentration ranging from 0.05% to about 0.1%. In some embodiments, the lipid-rich BSA composition is Albumax, and wherein the Albumax is present in the medium at a concentration ranging from 0.05% to about 0.5%. In some embodiments, the lipid-rich BSA composition is Albumax, and wherein the Albumax is present in the medium at a concentration ranging from 0.1% to about 0.5%. In some embodiments, the lipid-rich BSA composition is Albumax, and wherein the Albumax is present in the medium at a concentration ranging from 0.25% to about 0.5%.

In some embodiments, the medium comprises oxygen levels that are above levels that are considered to be low oxygen levels for the purposes of cell culture. In some embodiments, the medium comprises oxygen levels that are at least 1%. In some embodiments, the medium comprises oxygen levels that are at least 5%. In some embodiments, the medium comprises oxygen levels that are at least 10%. In some embodiments, the medium comprises oxygen levels that are at least 15%. In some embodiments, the medium comprises oxygen levels that are at least 20%. In some embodiments, the medium comprises oxygen levels that are considered to be normoxic levels for the purposes of cell culture (normoxia). In some embodiments, the medium comprises oxygen levels that are from about 20% to about 21%.

In accordance with the above media formulations, and as demonstrated by the results described herein, embodiments of the present disclosure also include a method for inducing and maintaining human trophoblast stem cells (hTSCs) from cytotrophoblasts (CTBs) ex vivo. In some embodiments, the method includes obtaining CTBs from placentas at birth, and culturing the CTBs in any of the media described herein for at least 3 passages, wherein the hTSCs express CDX2. Embodiments of the present disclosure also include an ex vivo human trophoblast stem cell (hTSC) derived from a cytotrophoblast (CTB) obtained from a placenta at birth, wherein the hTSC expresses CDX2.

Embodiments of the present disclosure also include a container comprising an ex vivo human trophoblast stem cell (hTSC) derived from a cytotrophoblast (CTB) obtained from a placenta at birth, wherein the hTSC expresses CDX2. In some embodiments, the container further comprising any of the media described herein.

### 3. MATERIALS AND METHODS

Culture of hESCs and hiPSCs. H1 and H9 hESCs and SC102A-1 hiPSCs were cultured on plates coated with vitronectin (5 μg/ml) at room temperature for at least one hour. Cells were cultured in 2 ml of TeSR-E8 medium at 37° C, in 5% CO₂ in 6-well plates and culture medium was replaced every day. When cells reached confluency, they were passaged using ReLeSR according to the manufacturer's protocol, at a 1:10 split ratio.

Differentiation of hESCs and hiPSCs to hTSCs. The day after passaging, hESCs or human induced pluripotent stem cells (hiPSCs) were differentiated by treatment with CYM5541 (2 μM), SB431542 (25 μM), BMP4 (20 ng/ml) in TeSR-E7 for 2 days for HI hESCs and 3 days for H9 hESCs and SC102A-1 hiPSCs. The medium was replaced every day. After 2 or 3 days of treatment, cells were dissociated with TrypLE for 5 minutes at 37° C. All cells were seeded in a 6-well plate pre-coated with 3 μg/ml of vitronectin and 1 μg/ml of Laminin 521 at a density of ˜5×10⁴ cells per well and cultured in 2 ml of DTM7 medium.

hTSCs from placenta-derived CTBs. Placenta-derived CTBs-CT30, CT27 (female) and CT29 (male), a kind gift from Drs. Hiroaki Okae and Takahiro Arima (Tohoku University. (Okae et al., 2018))—were grown in a 6-well plate pre-coated with 3 μg/ml of vitronectin and 1 μg/ml of Laminin 521 at a density of ˜5×10⁴ cells per well and cultured in 2 ml of TSCM developed by Okae et al. (2018) (DMEM/F12 supplemented with 0.1 mM 2-mercaptoethanol, 0.2% FBS, 0.5% Penicillin-Streptomycin, 0.3% BSA, 1% ITS-X supplement, 1.5 μg/ml L-ascorbic acid, 50 ng/ml EGF, 2 μM CHIR99021, 0.5 μM A83-01, 1 μM SB431542, 0.8 mM VPA and 5 μM Y27632). These cells were directly passaged into DTM7 for formation of hTSCs; complete transition took about 5 passages. Cells passaged into DTME medium were assessed after at least about 2 passages, and cells passaged into TSCMUA were assessed after at least about 5 passages.

Differentiation of term CTBs to hTSCs. Term CTBs that were previously frozen were thawed directly into DTM7 medium, or TSCMUA medium (see, e.g., Table 1). To assess DTME medium, cells cultured in TSCMUA were passaged into DTME.

Culture of hTSCs. hTSCs were cultured in 2 ml of DTM7 at 37 C in 5% CO₂. Culture medium was replaced every 2 days. When hTSCs reached 70-90% confluence, they were dissociated with TrypLE at 37° C. for 5-10 minutes and passaged to a new 6-well plate pre-coated with 3 μg/ml of vitronectin and 1 μg/ml of Laminin 521 at a 1:3-1:4 split ratio and were supplemented with Y-27632 upon passage to aid in single cell attachment. Cells were routinely passaged approximately every 4-6 days. hTSCs at passages 2+ (typically passage 5+) were used for analysis.

Immunofluorescence analysis. For immunofluorescence analysis, cells were grown on glass-bottom culture dishes coated with 3 μg/ml vitronectin and 1 μg/ml of Laminin 521. Cells were fixed either using 4% paraformaldehyde in PBS for 10 min, permeabilized with 0.5% Triton X-100 for 5 min and blocked in 3% BSA/PBS with 0.1% human IgG and 0.3% Triton X-100 for 1 hr. Cells were then incubated overnight with the primary antibody diluted in blocking buffer. The following primary antibodies were used: anti-KRT7 (SCB, 1:50), anti-KRT7 (CST, 1:500), rabbit anti-hCG (1:100), mouse anti-hCG (1:100), anti-YAP (1:200), anti-TFAP2C (1:400), anti-P63 (1:600), anti-GATA3 (1:500), anti-TEAD4 (1:250), anti-CDX2 (1:300), anti-VE-Cadherin (1:400), anti-HLA-G (1:300), anti-syncytin (1:50). Corresponding isotype controls (rabbit polyclonal IgG, rabbit XP IgG, mouse IgG1, and mouse IgG2a) were used at primary antibody concentrations. Alexa Fluor 488- or Alexa Fluor 647-conjugated secondary antibodies were used as secondary antibodies. Nuclei were stained with DAPI and all samples were imaged using a Zeiss LSM 710 or 880 laser scanning confocal microscope (Carl Zeiss, Germany).

hTSC cell culture. hTSCs were cultured as previously described by Okae et al. with minor modifications. Additionally, similar protocols were used for the TSCMUA, DMT7, and DTME media described further herein. Cells were cultured in 2 mL of TSCM medium (Dulbecco's Modified Eagle Medium/Nutrient Mixture F-12 (DMEM/F-12) supplemented with 0.1 mM 2-mercaptoethanol, 0.2% FBS, 0.5% Penicillin-Streptomycin (Pen/Strep), 0.3% BSA, 1% Insulin-Transferrin-Selenium-Ethanolamine (ITS-X), 1.5 μg/mL L-ascorbic acid, 50 ng/mL EGF, 2 μM CHIR99021, 0.5 μM A83-01, 1 μM SB431542, 0.8 mM VPA, and 5 μM Y-27632) at 37° C., and 5% CO_{2,} on 35 mm polystyrene plates, pre-coated with 3 μg/ml of vitronectin and 1 μg/ml of Laminin 521. Culture medium was replaced every two days. When cells reached confluence, they were dissociated with TrypLE Express for 10-15 minutes at 37° C., and passaged at a 1:10 split ratio. Cells were routinely passaged approximately every 4-6 days. All hTSCs used in this study were passaged at least 5 times prior to use in experiments.

EVT and STB differentiation. Prior to differentiation. hTSCs at confluence were dissociated into single cells using TrypLE Express and 1.5×10⁵ cells were seeded onto a new 35 mm polystyrene or glass plate pre-coated plate with 3 μg/ml of vitronectin and 1 μg/ml of Laminin 521. For STB differentiation, cells were cultured in defined trophoblast differentiation medium (DTDM) (DMEM/F-12 supplemented with 1% ITS-X, 75 μg/mL L-ascorbic acid). For EVT differentiation, DTDM was supplemented with 150 μg/mL laminin-1 after cells were plated in DTDM. 5 μM Y-27632 and 50 ng/mL EGF was added at passage. Cell culture medium was replaced every 2 days and cultures were analyzed at day 6 unless otherwise specified. 7.5 μM A83-01 was used where specified. 2-Step EVT and STB using forskolin were differentiated as previously described, with some minor modifications. Briefly, 1.5×10⁵ cells were passaged and seeded onto a 35 mm polystyrene or glass plate pre-coated plate with 3 μg/ml of vitronectin and 1 μg/ml of Laminin 521. For EVT differentiation, cells were cultured in EVTM (DMEM/F12 supplemented with 0.1 mM 2-mercaptoethanol, 0.5% Penicillin-Streptomycin, 0.3% BSA, 1% ITS-X supplement, 100 ng/ml NRG1, 7.5 μM A83-01, 2.5 μM Y27632, and 4% KSR). Matrigel was added to a final media concentration of 2% after suspending the cells in EVT medium. On day 3, the medium was replaced with the EVT medium without NRG1 and Matrigel was added to a final concentration of 0.5%. EVTs were fixed on day 6. For STB differentiation, cells were cultured in STBM (DMEM/F12 supplemented with 0.1 mM 2-mercaptoethanol, 0.5% Penicillin-Streptomycin, 0.3% BSA, 1% ITS-X supplement, 2.5 μM Y27632, 2 μM forskolin, and 4% KSR). Media was replaced on day 3 and cells were fixed on day 6.

Immunostaining. For immunofluorescence analysis, 3×10⁴ cells were grown on 24-well glass bottom plates coated with 3 μg/ml of vitronectin and 1 μg/ml of Laminin 521. After six days, cells were fixed with 4% paraformaldehyde fixative solution for 5 minutes, permeabilized with 0.5% Triton X-100 in PBS for 10 minutes, then blocked in blocking buffer (0.5% BSA, and 200 μM human IgG in PBS) for at least one hour. Cells were then incubated overnight at 4° C, in primary antibody diluted in blocking buffer. Primary antibodies used were anti-HLA-G (1:250), anti-Notch1 (1:200), anti-hCG (1:50), anti-SDC-1 (1:250), anti-KRT7 (1:50), anti-p63 (1:50), anti-TEAD4 (1:50), anti-CDX2 (1:250), anti-GATA3 (1:500), anti-YAP (1:200), anti-AP-2γ(1:333), anti-Oct4 (1:200), anti-HIF1α (1:100), and anti-HIF2α (1:100). Secondary antibodies were added an hour before imaging. Corresponding isotype controls (rabbit monoclonal IgG, rabbit XP IgG, mouse IgG1, mouse IgG2a, and mouse IgG2b) were used at primary antibody concentrations. Alexa Fluor 488- or Alexa Fluor 647-conjugated secondary antibodies were used. Nuclei were stained with DAPI and images were taken with a laser scanning confocal microscope (LSM880, Carl Zeiss, Germany).

Confocal image analysis. Image analysis was conducted using an image processing algorithm created in MATLAB R2021a. All image processing was performed post hoc. DAPI (blue channel) was isolated from the RBG image, binarized, and processed to accurately represent the number of cells in each image. Two images with known cell number were used to develop the processing steps and these were then extrapolated to all other images. The primary antibody stain of interests (red and green) was isolated and processed in the same manner. The average intensity of the red and green stains nearest each cell was assigned as the average expression intensity for that cell. If the nearest red or green stain was farther than the nearest blue stain, then the cell was assigned the average isotype control expression value. This was performed for 14 isotype control images and 14 experimental images (7 images for each of two replicates). For analysis labeled nuclear, only pixels that overlapped DAPI pixels were used for average expression intensity. Data was normalized by the average isotype control expression intensity. The code used for image analysis can be found at github.com/vkarakis/2D_IF_analysis.git.

Statistical analysis. For immunofluorescence analysis, statistical analysis was conducted using the non-parametric Mann-Whitney U test because the data is not normally distributed. The analysis was performed in Microsoft Excel using the test for large sample size. Results of this test are given as a p-value to compare differences in medians. Statistical significance was inferred at p<0.05.

Membrane staining. hTSCs and STB were cultured as previously described or by Okae et al. Cells were washed and subsequently incubated with 1-2 μM Di-8-ANEPPS and DAPI on ice for at least 1 hour. Cells were washed once and imaged in FluoroBrite™ DMEM using a Keyence BZ-X810 system.

### 4. EXAMPLES

Considering restrictions on research with human embryos and fetal tissues, human trophoblast stem cells (hTSCs) derived from six-to eight-week placentas or blastocysts have gained prominence as a powerful model for in vitro studies on early placental development. Like the epithelial cytotrophoblast (CTB) in the first trimester of gestation, hTSCs can differentiate to form extravillous trophoblasts (EVTs) and the multinucleate syncytiotrophoblast (STB). However, these hTSCs could not be derived from term cytotrophoblasts. As described further herein, experiments were conducted to investigate how the addition of UK-5099 and AlbuMAX to the trophoblast stem cell medium (TSCM) leads to the formation of hTSCs derived from term CTB. These hTSCs express similar CTB markers and can further differentiate to either the EVT or the STB lineage. Unlike hTSCs derived from first trimester CTBs, hTSCs derived from term express CDX2, a marker indicative of the trophoblast stem cell niche in vivo.

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

Derivation of CDX2⁺ hTSCs from the placenta at birth using a medium with defined composition. Previously results have demonstrated the culture of CDX2⁺ hTSCs derived from pluripotent stem cells in TM4 medium (Mischler, A, et al., Two Distinct Trophectoderm Lineage Stem Cells from Human Pluripotent Stem Cells. Journal of Biological Chemistry 2021, 296, 100386. (doi.org/10.1016/j.jbc.2021.100386)). However, this previous experimental approach was unable to culture CT29 and CT30 hTSCs derived by the methods of Okae et al, in TM4 medium. Therefore, multiple additional culture conditions were screened and various medium compositions were identified (e.g., TSCMUA, DTM7, and DTME) that supports derivation of CDX2⁺ hTSCs from CTBs of the placenta obtained at birth. The details of these conditions and media comparisons are shown below in Table 1.

Table 1: Comparison of DTM7 medium with other related media that differ in some components. ^{*}Concentrations of glucose, pyruvate, and ascorbic acid are relative to that used in Okae et al. ⁺Some variability between cell lines and between experiments is observed and the medium is not robust for maintenance of hTSCs relative to DTME medium. *Although these conditions were not tested, conditions that support culture of CDX2+hTSCs from term placentas also support culture of CDX2+ hTSCs from first trimester placental samples and pluripotent stem cells. A “checkmark” indicates present; an “X” indicates not present.

| | Okae | | | | | |
|---|---|---|---|---|---|---|
| | et al. | | | | | |
| Component | (TSCM) | TSCMUA | TM4 | DTM5 | DTM7 | DTME |
| BSA/Serum | ✓ | ✓ | X | X | X | ✓ (BSA) |
| β-ME | ✓ | ✓ | X | X | X | X |
| EGF | ✓ | ✓ | X | X | X | ✓ |
| FGF10 | X | X | ✓ | ✓ | ✓ | ✓ |
| HGF | X | X | X | ✓ | ✓ | X |
| CHIR99021 | ✓ | ✓ | ✓ | ✓ | ✓ | ✓ |
| A83-01 | ✓ | ✓ | ✓ | ✓ | ✓ | ✓ |
| nicotinamide | X | X | X | ✓ | ✓ | ✓ |
| lactate | X | X | X | X | ✓ | X |
| S1PR2 agonist | X | X | X | X | ✓ | X |
| S1PR3 agonist | X | X | ✓ | X | X | X |
| SB431542 | ✓ | ✓ | X | X | X | X |
| VPA | ✓ | ✓ | X | X | X | X |
| Y27632 | ✓ | ✓ | X | X | X | X |
| Decanoic acid | X | X | X | X | X | ✓ |
| Dimethyl | X | X | X | X | X | ✓ |
| alpha-ketoglutarate | | | | | | |
| Lysophosphatidic | X | X | X | X | X | ✓ |
| acid (LPA) | | | | | | |
| Albumax II | ✓ | ✓ | X | X | X | X |
| UK5099 | ✓ | ✓ | X | X | X | X |
| Glucose* | 1x | 1x | 1x | 0.4x | 0.4x | 0.4x |
| Pyruvate* | 1x | 1x | 1x | 2x | 2x | 2x |
| ascorbic acid* | 1x | 1x | 43x | 50x | 50x | 50x |
| Ability of conditions to | Okae | | | | | |
| support CDX2+ hTSCs | et al. | | | | | |
| in culture | (TSCM) | TM4 | TSCMUA | DTM5 | DTM7 | DTME |
| Supports CDX2⁺ | No | Yes | Not tested | Yes⁺ | Yes | Not tested |
| hTSCs from pluripotent | | | (expected | | | (expected |
| stem cells | | | yes)* | | | yes)* |
| Supports CDX2⁺ | No | No | Not tested | Yes⁺ | Yes | Not tested |
| hTSCs from CTBs from | | | (expected | | | (expected |
| first trimester placentas | | | yes)* | | | yes)* |
| Supports CDX2⁺ | No | No | Yes | No | Yes⁺ | Yes |
| hTSCs from CTBs from | | | | | | |
| term placentas | | | | | | |

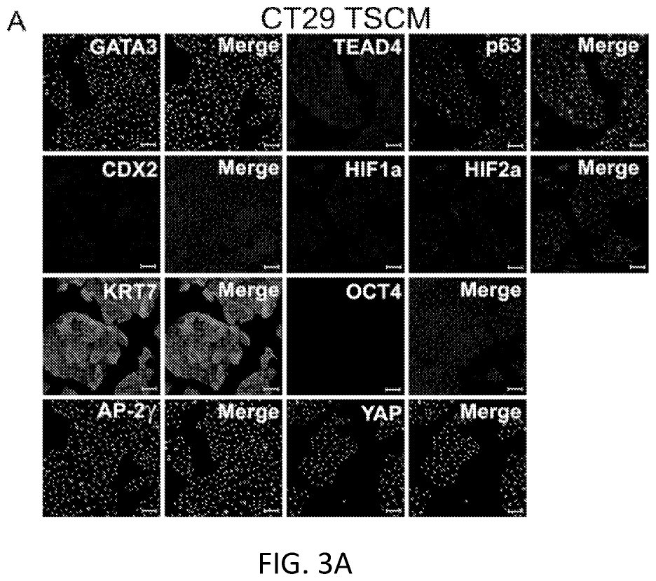

TSCMUA. hTSCs were originally derived from blastocyst-stage embryos and first trimester placental samples in a medium referred to herein as TSCM. TSCM does not support the derivation of hTSCs from term placentas (placentas at birth). A recent study by Wang et al. claimed that hTSCs can be derived from term placentas using TSCM if cells are cultured at 1% oxygen. However, it is important to note that cells cultured at 1% oxygen in TSCM do not express CDX2. RNAseq comparison of CT30 hTSCs (derived by Okae et al. from first trimester placentas in TSCM) and hTSCs from term placentas cultured in 1% oxygen using published data showed that there is no statistically significant difference in CDX2 transcript expression between the two conditions. Further, very low transcript abundance is found. Note that hTSCs derived by Okae et al. from first trimester placentas (CT30 and CT29 hTSCs) do not show protein expression of CDX2 (FIGS. 3A-3B).

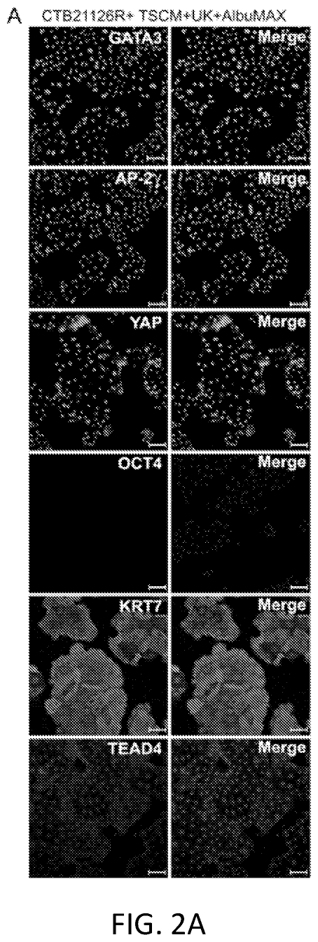

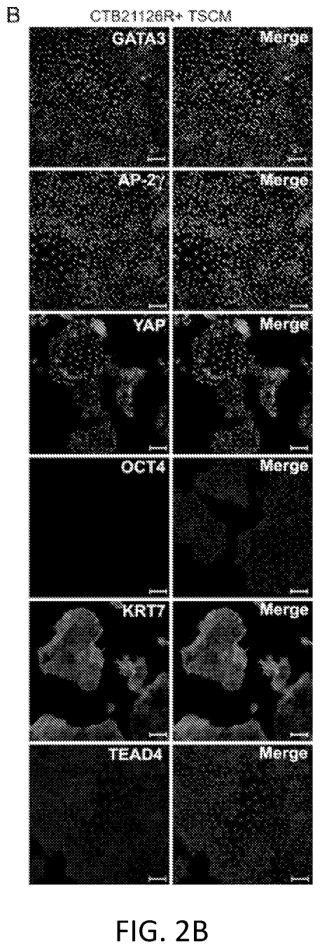

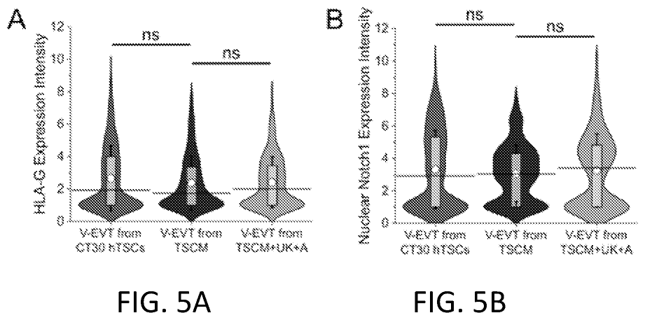

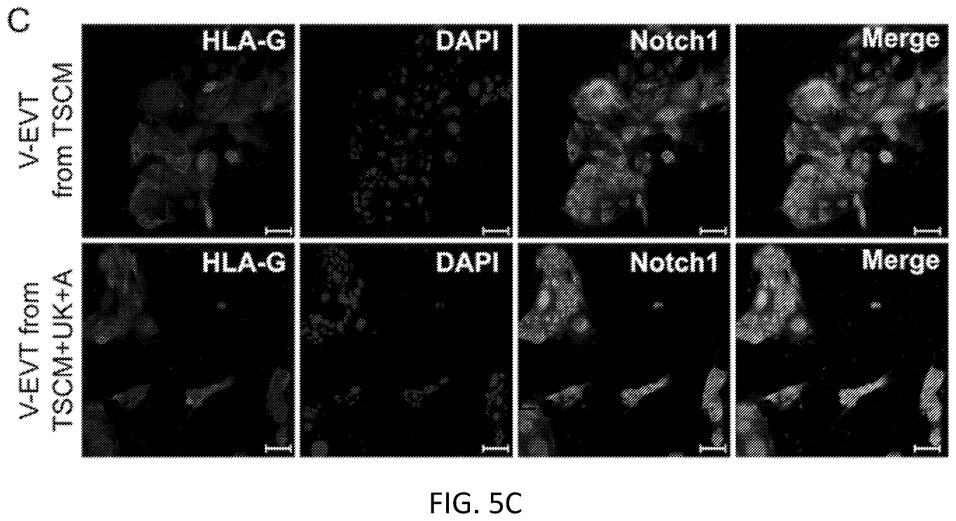

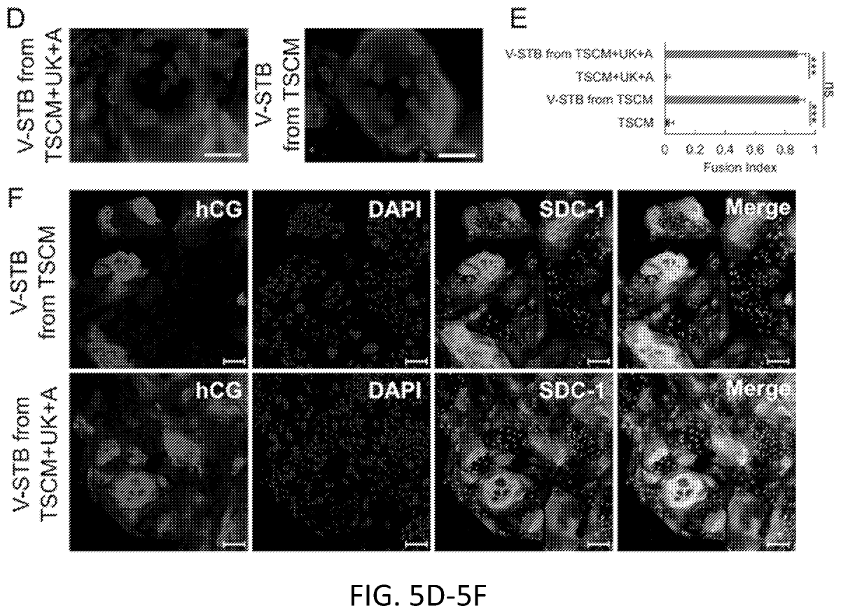

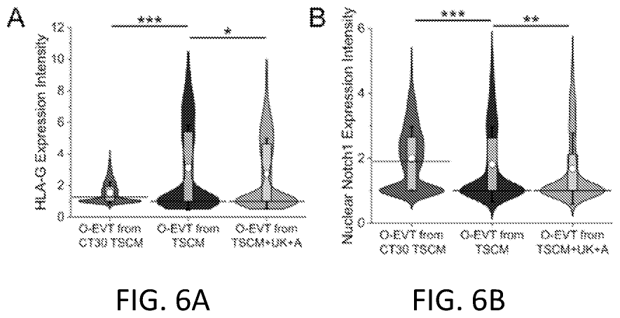

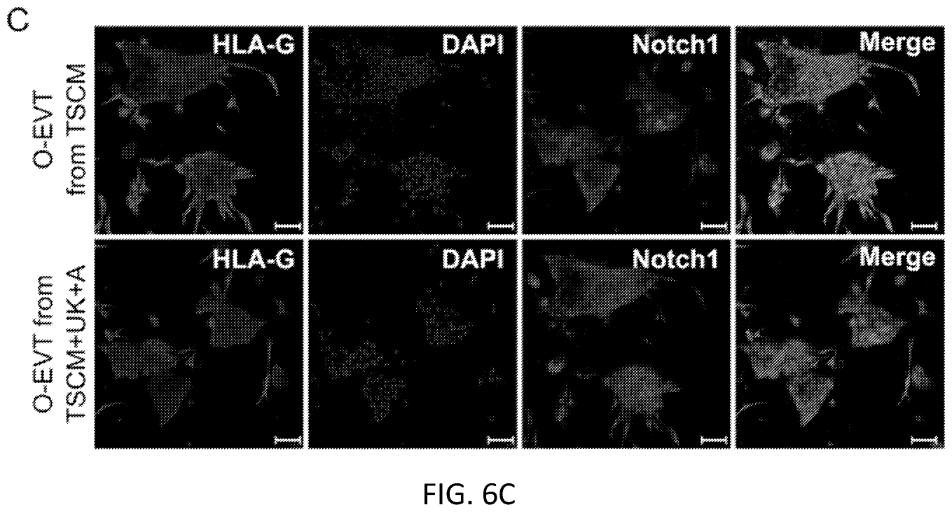

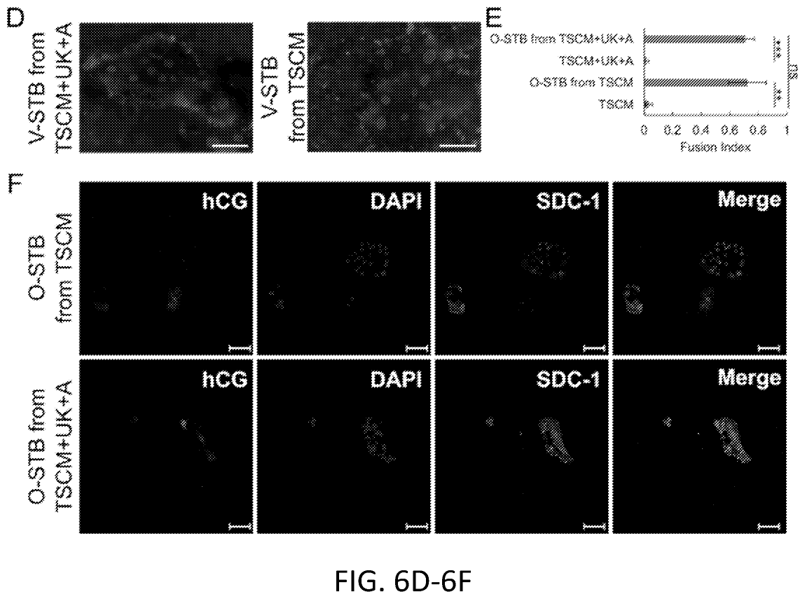

Results of the present disclosure demonstrate that CDX2⁺ hTSCs can be derived from term placentas in TSCMUA medium, which includes the mitochondrial pyruvate uptake inhibitor UK5099 (50 nM) and Albumax II (0.2%). These cells exhibit morphology similar to hTSCs derived from first trimester placentas, and express other hTSC markers including YAP. GATA3, TFAP2C (AP2γ) and TEAD4, and the pan-trophoblast marker KRT7 (FIG. 2A). Further, these hTSCs can differentiate to syncytiotrophoblast (STB) and extravillous trophoblast (EVT), characteristic of hTSCs (FIGS. 5 and 6). Additionally, EVTs express the characteristic markers HLA-G and Notch1 upon differentiation with two different protocols; STB are multinucleate and express the markers SDC-1 and hCG when hTSCs are differentiated as previously described.

AlbuMAX is lipid-rich bovine serum albumin (BSA) that is commercially available. The exact lipid composition of AlbuMAX is not known; however, lipids constitute ˜0.65% by dry weight of AlbuMAX. Free fatty acids and phospholipids are key components of AlbuMAX. Previous work has shown that the phospholipids sphingosine-1-phosphate and lysophosphatidic acid can mediate at least in part the effect of AlbuMAX; on the other hand, free fatty acids can also activate G-protein coupled receptor signaling. Therefore, experiments were conducted to determine if AlbuMAX could be replaced by supplementation with lipid-free albumin (BSA) along with phospholipids or free fatty acids. Specifically, experiments were conducted to determine if AlbuMAX could be replaced by BSA+LPA or BSA+chemically defined lipid concentrate (CDL); CDL is a commercially available mix containing free fatty acids. However, results indicated that cells cultured under these conditions are unable to differentiate to extravillous trophoblasts or syncytiotrophoblast, as assessed by expression of the EVT markers HLA-G and Notch1, and the STB markers SDC-1 and hCG. Similarly, differentiation potential is also compromised if UK5099 is removed from TSCMUA. Thus, the composition of TSCMUA is important.

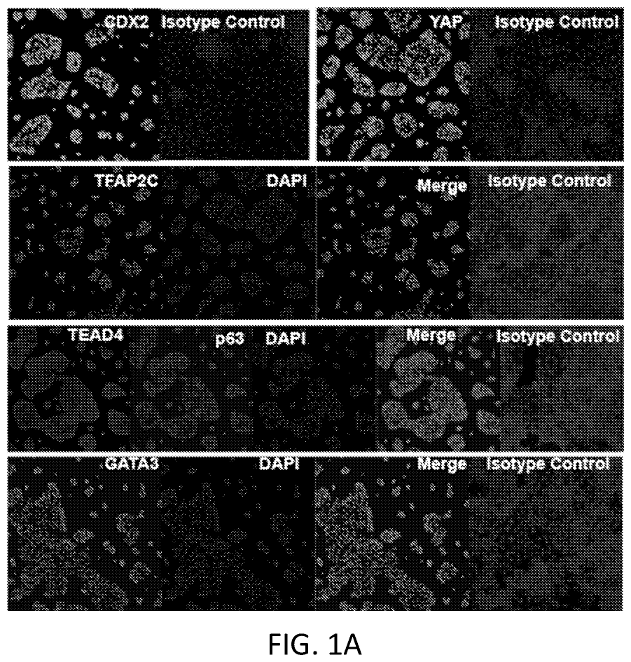

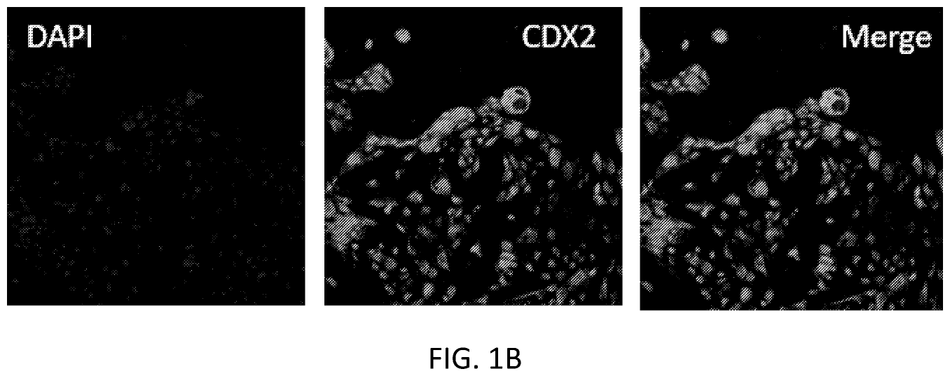

The exact lipid composition of AlbuMAX, and therefore TSCMUA, is poorly defined. Therefore, experiments were conducted to develop two chemically defined media formulations—DTM7 and DTME—for culture of CDX2⁺ hTSCs from term placentas. CTBs cultured in DTM7 medium exhibit morphology similar to hTSCs derived from first trimester placentas. Further, these cells express CDX2, in addition to other hTSC markers including YAP, TFAP2C, p63, TEAD4 and GATA3 (FIGS. 1A-1B). Here, CDX2, YAP, TFAP2C and GATA3 were stained individually; TEAD4 and p63 were co-stained. Similarly, cells cultured in DTME express CDX2 (FIGS. 1A-1B).

These results underscore that the composition of the chemically defined media is important. For instance, when the concentration of glucose used in DTM7E is equivalent to that used in TSCMUA and a low concentration of UK5099 is included (i.e., high glucose DTM7E+UK5099), CDX2 expression is not obtained and the cells lose their ability to differentiate to EVT and STB. In some cases, LPA can be substituted by S1P or other small molecule agonists of the S1P receptor (as seen by their usage in DTM7). Also, a cell-permeable version of alpha-ketoglutarate (DMKG) can also be used. Concentrations in DTME were as followed: ascorbic acid at 75 micrograms/mL; CHIR99021 at 2 micromolar; A83-01 at 533 nM; BSA at 0.3%; FGF10 at 37 ng/mL; EGF at 50 ng/mL; decanoic acid at 500 nM; DMKG at 4 mM; and nicotinamide at 10 mM.

### Example 2

Defined Trophoblast Medium (DTM7 and DTME). As described further herein, a chemically defined cell culture medium for inducing and maintaining human trophoblast stem cells (hTSCs) from cytotrophoblasts (CTBs) from placentas at birth was developed (i.e., DTM7 and DTME). In one embodiment of the present disclosure, the DTM7 composition comprises the following: about 22 mL low glucose DMEM w/GlutaMAX (Thermo Fisher, 10567014); about 22 mL F/12 w/GlutaMAX (Thermo Fisher, 31765092); about 450 μL ITS-X (1%, PeproTech 00-101); about 112 μL L-ascorbic Acid (30 mg/mL, final conc: 75 μg/mL, Sigma, A8960); about 45 μL HGF (50 μg/mL, final conc: 50 ng/mL, Human Recombinant Hepatocyte Growth Factor, Stem Cell Technologies 78019); about 12 μL A83-01 (2 mM, final conc: 2 μM, Tocris, 2939); about 45 μL CHIR99021 (2 mM, final conc: 2 μM, Tocris, 4423); about 33 μL FGF10 (50 μg/mL, final conc: 36 ng/mL Stem Cell Technologies, 78037.1); about 450 μL nicotinamide (1 M, final conc: 10 mM, Sigma, N3376); about 112 μL CYM5520 (2 mM, final conc: 5 μM, Sigma, SML0680); and about 112 μL Sodium L-lactate (4 M, final conc: 10 mM, Sigma, 71718). As would be recognized by one of ordinary skill in the art based on the present disclosure, certain substitutions of one or more of the above components of DMT7 can be made in the compositions and methods described herein.

Stem Cell Culture. Embodiments of the present disclosure also include methods for culturing and maintaining hTSCs from CTBs. For example, the following protocol can be used with DTM7 medium:

Passaging: (1) Coat 35 mm plastic plate (or 6-well) (Sarstedt, 83.3900) with 5 μg/ml of vitronectin ((400 μL, Thermo Fisher Cat #A14700) and 1 μg/ml of Laminin 521 (200 μL, Stem Cell Tech Cat #77003). Can add each matrix separately and swirl on plate to mix. Leave for 1 hour at RT or overnight in 4° C. (2) Wash plate with PBS w/o Ca/Mg. (3) Add 700 μL TrypLE (Thermo Fisher, 12604021) and incubate at 37° C. for 10 min. (4) Add 700 μL normal D/F12 to a 1.5 mL microcentrifuge tube. (5) After 10 min, gently pipette around the plate to lift off all cells. Add the 700 μL of cells in TrypLE to the tube with the D/F12 and pipette to mix. (6) Centrifuge at 370 rcf for 5 min. (7) Remove media and resuspend the pellet in DTM7. (8) Add 2 mL of DTM7 to pre-coated plate. (9) Add 2 μL of Y27632 (5 mM stock, final conc: 5 μM, Tocris, 1254) and gently swirl to mix (can add Y27632 at passage instead of when changing media). (10) Add cells to media (plate 1:2 while transitioning and 1:5 after). On days 2 and 4, replace the medium with DTM7 (no added Y27632). Methods for culture of cells in DTME are similar.

When transitioning from trophoblast stem cell medium (TSCM) described by Okae et al, to DTM7, for ˜5 passages, plate at 1:2 or 1:3. Confluency should be kept high to ease with the transition. After 5 passages, start to passage 1:5 or even 1:10 like TSCM cells; however, if passaging at 1:10, the cells may not reach confluency by day 6 (growth rate is slower than TSCM). Even if the cells are not confluent, passaging should occur by at least day 6 (depending on health of cells). Usually by day 6, the cells stop proliferating out on the plate and start proliferating up on top of a colony, which indicates that passaging may be needed. If cells are confluent before day 6, passaging can occur.

### Example 3

Derivation of hTSCs from term CTBs using TSCM supplemented with AlbuMAX and UK5099. Isolated term cytotrophoblast cells were thawed and cultured in trophoblast stem cell media (TSCM) as previously described, supplemented with UK5099 (UK) and AlbuMAX (A). These cells grew slowly for the first few passages before beginning to adopt the hTSC morphology. These term hTSCs expressed hTSC markers GATA3, AP-2y, YAP, KRT7and TEAD4, and were negative for OCT4 expression (FIG. 2A). Similarly, hTSCs from first trimester placental samples expressed these same markers (FIGS. 3A-3B). After 5 passages, AlbuMAX and UK5099 could be dropped from the medium and cultured in TSCM. These cells also expression the same markers, GATA3, AP-2γ, YAP, KRT7, TEAD4, and were negative for OCT4 (FIG. 2B).

### Example 4

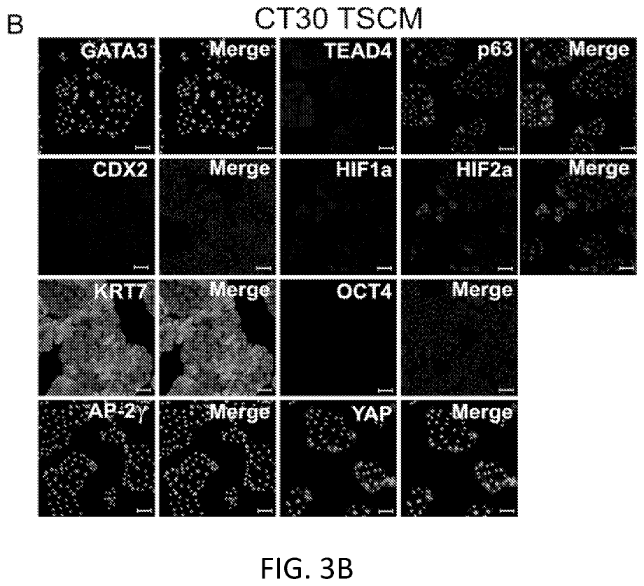

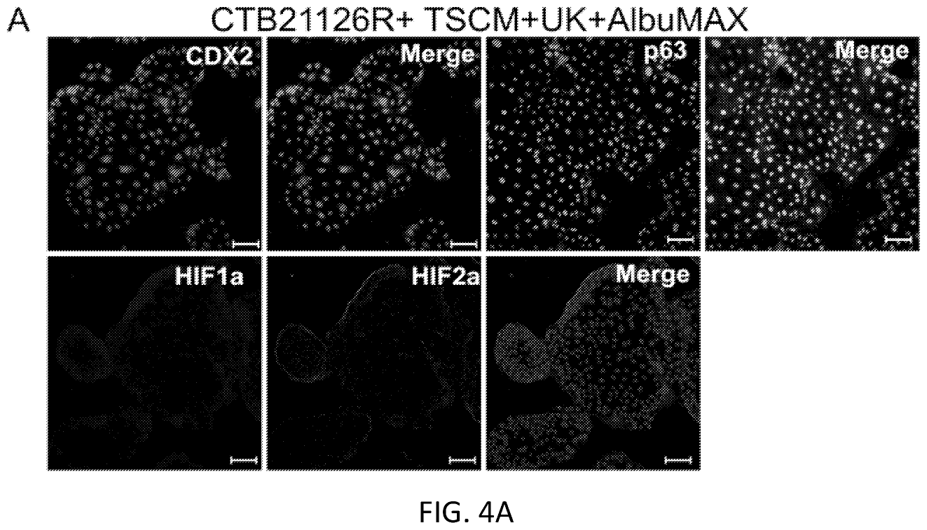

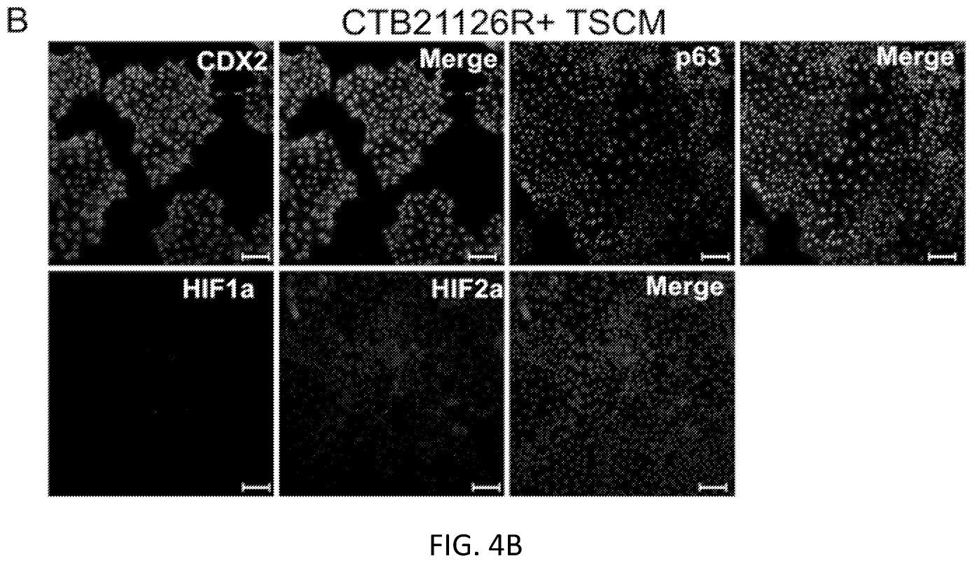

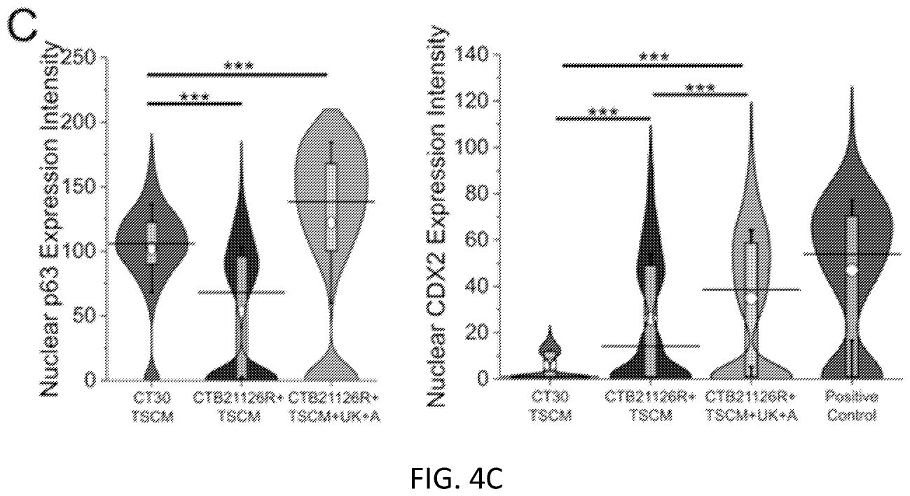

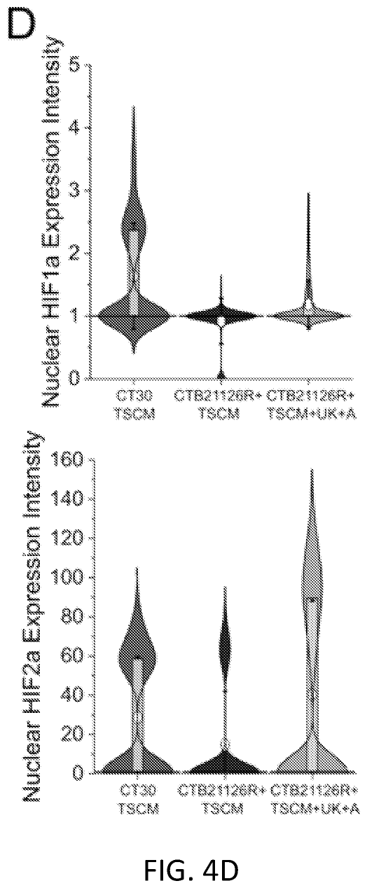

hTSCs from term CTB gain expression of CDX2. hTSCs from term cells also expressed another relevant CTB marker, p63 and did not express HIF1α or HIF2α (FIGS. 4A-4D). Importantly, these term cells expressed the CTB marker, CDX2 as well. Quantitative analysis was performed to compare relative expression of p63, HIF1α, and HIF2α to hTSCs derived from first trimester placenta samples (CT30). CT30 hTSCs and CTB21126R+ hTSCs cultured in either medium were all positive for p63 expression (FIG. 4C). Interestingly, however, hTSCs from term CTBs were also positive for CDX2, unlike hTSCs derived from first trimester CTBs (FIG. 4C). Interestingly, CDX2 expression decreased after hTSCs were switched to the TSCM maintenance condition, implying that AlbuMAX and UK5099 are important for CDX2 expression (FIG. 4C). HIF1α and HIF2α on the other hand, were negative in CT30 hTSCs as well as the term hTSC cell lines, with little to no expression seen (FIG. 4D).

### Example 5

hTSCs from term CTB differentiate to EVT and STB using the 1-Step protocol. Experiments were conducted to determine how these hTSCs derived from term CTBs would differentiate to either EVT or STB. EVT and STB differentiation were performed using the protocol described previously. EVTs expressed EVT markers, HLA-G and Notch1 and STB expressed STB markers hCG and SDC-1 from both TSCM and from TSCMUA (FIGS. 5C and 5F). Quantitative analysis was performed on EVTs in these two conditions and compared it to CT30 hTSCs isolated from first trimester CTBs and found that both HLA-G and Notch1 expression intensity differences were not significant in either HLA-G or Notch1 expression (FIGS. 5A and 5B). For STB differentiation, a membrane stain was also performed and it was found that STB had significant cell fusion from TSCM or TSCM+UK+A (FIG. 5D) with a fusion index significantly higher than the hTSC control (FIG. 5E). The calculated fusion index is similar to what was previously seen in CT30 and CT29 STB from first trimester hTSCs.

### Example 6

hTSCs differentiate to EVT and STB using the Okae protocol. Experiments were also conducted to determine if these hTSCs would have difficulty differentiating to EVT and STB using the differentiation technique described by Okae et al. Unlike EVTs from the previous method, EVTs expressed significantly lower levels of Notch1 than EVTs formed from first trimester placentas (FIG. 6B). Additionally, while the mean HLA-G expression was higher than hTSCs derived from first trimester placentas, the median HLA-G expression was much lower (FIG. 6A). This, along with evidence from the immunofluorescence staining (FIG. 5C), suggests that the difference in HLA-G expression intensity between cells in colonies versus cells beginning to undergo EMT is greater in term cell than from first trimester cells. For STB differentiation, while significant cell death was present with low hCG and SDC-1 staining (FIG. 6F), cell fusion was also observed with a membrane stain in both conditions (FIG. 6D), with a fusion index significantly higher than the hTSC control (FIG. 5E). The calculated fusion index is similar to what was previously seen in CT30 and CT29 STB from first trimester hTSCs using this differentiation method.

### Example 7

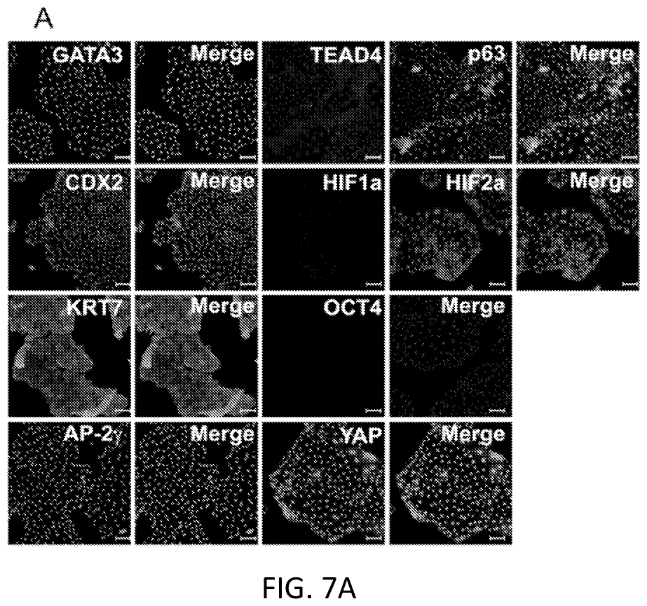

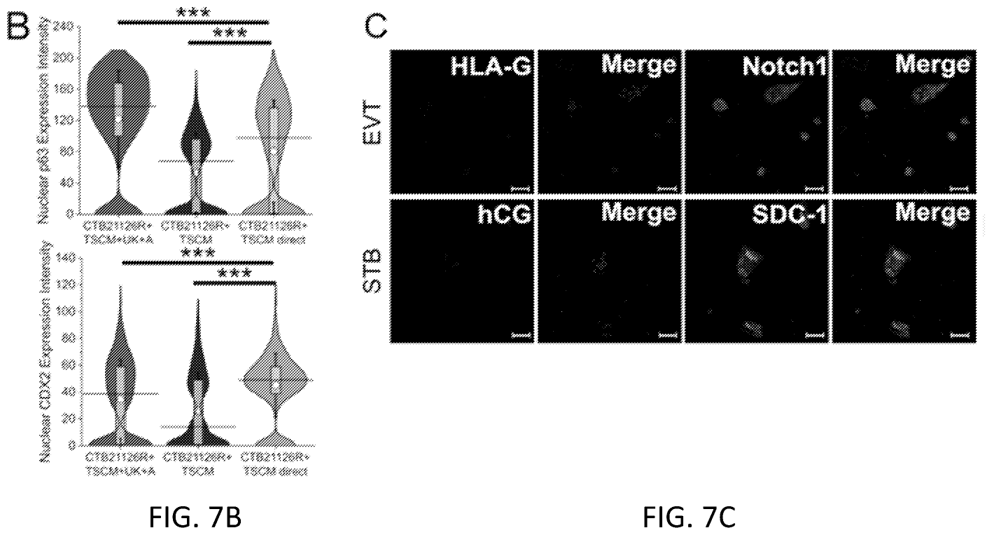

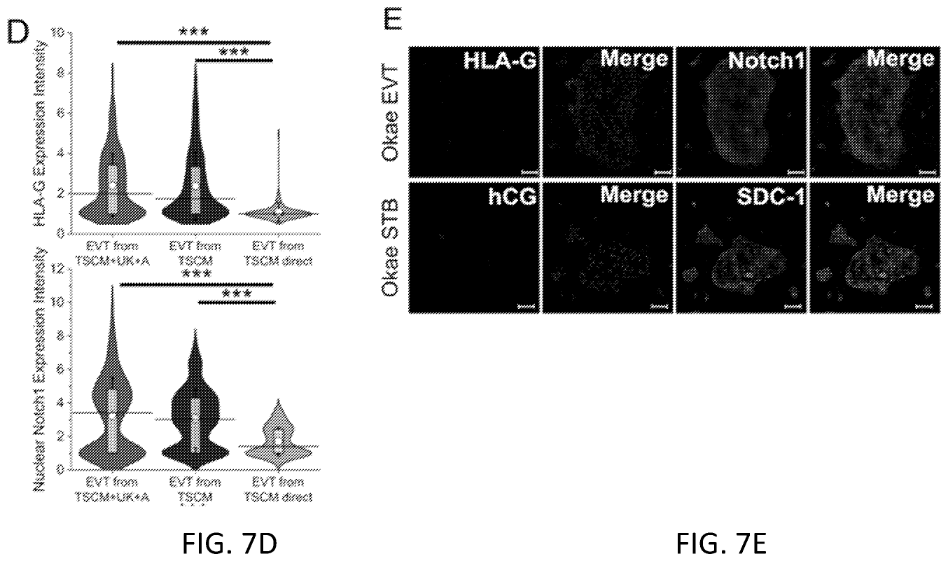

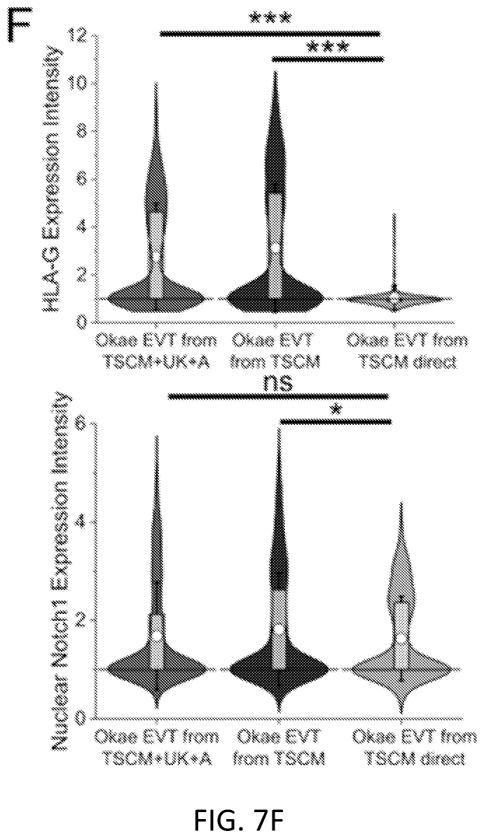

hTSC derivation from term CTB does not occur in TSCM alone. Because hTSCs were able to be transferred from TSCMUA to TSCM in culture, experiments were conducted to investigate whether CTBs can be thawed directly in TSCM without having to transfer cultures. It was found that hTSCs thawed directly into TSCM alone did, in fact, grow and proliferate. These cells expressed relevant CTB markers, GATA3, TEAD4, p63, KRT7, AP-2γ, and YAP (FIG. 7A). Additionally, these cells also exhibited CDX2 expression, low HIF2α and no HIF1α expression, and were negative for Oct4 (FIG. 7A). Quantitative analysis revealed positive nuclear p63 staining though there was more cytoplasmic staining observed compared to TSCM and TSCM+UK+AlbuMAX (FIGS. 7A and 7B). Interestingly, however, CDX2 expression was significantly higher than the prior two cultures (FIG. 7B). However, when these cells were differentiated, EVT or STB were not formed (FIGS. 7C-7F). Cells differentiated towards EVT did not express HLA-G, nor nuclear Notch1 using either differentiation protocols and cells differentiated towards STB did not express hCG nor SDC-1 (FIGS. 7C-7F).

### Example 8

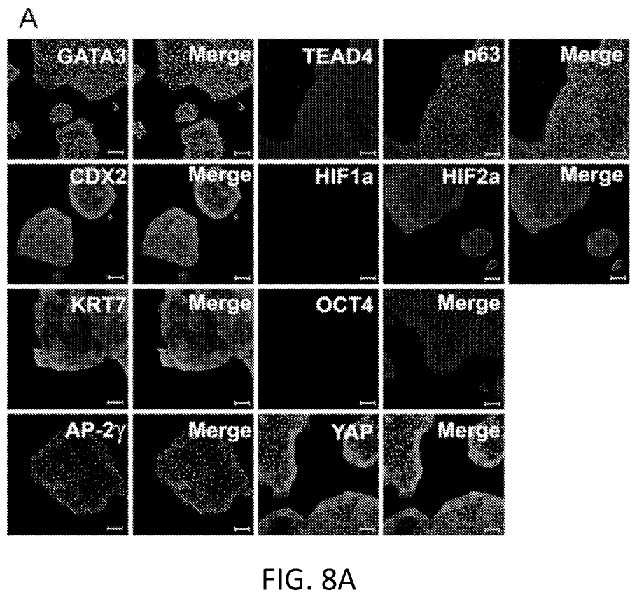

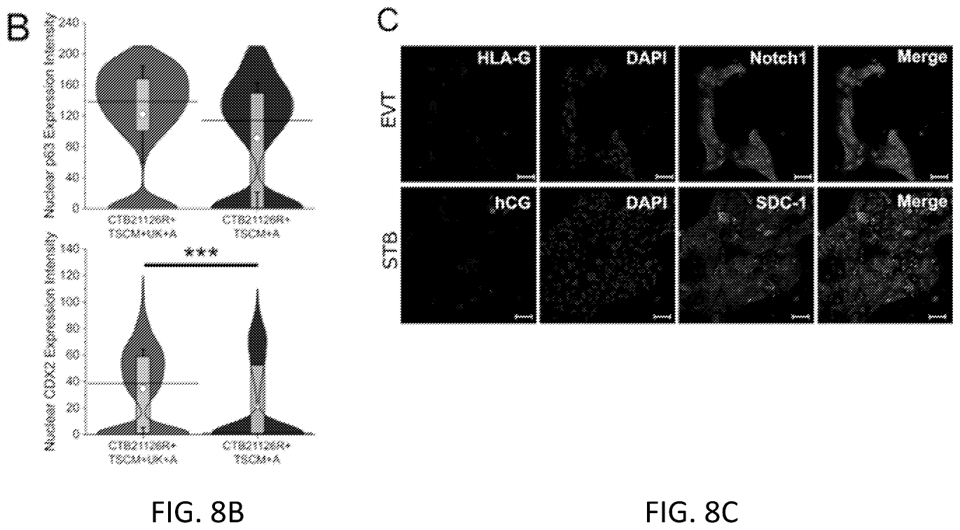

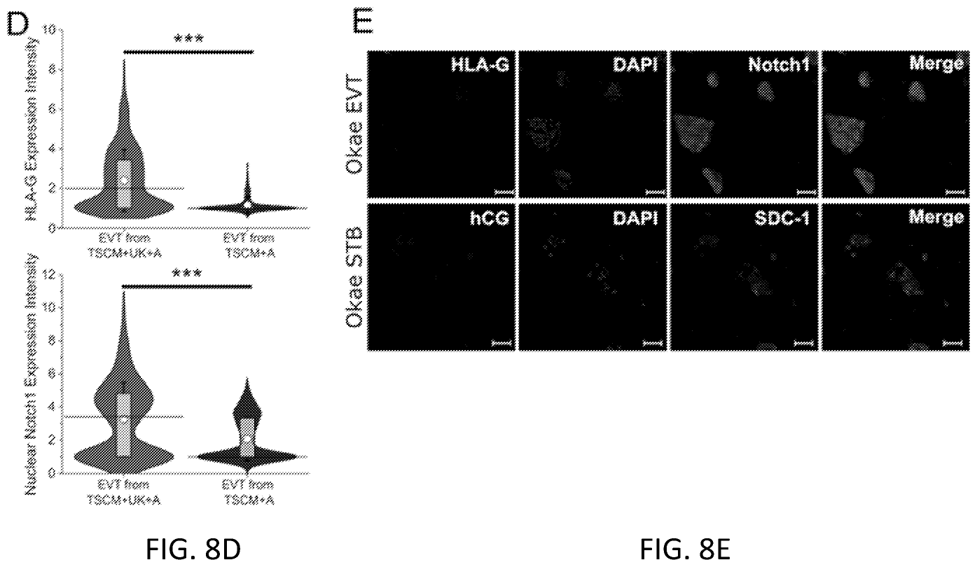

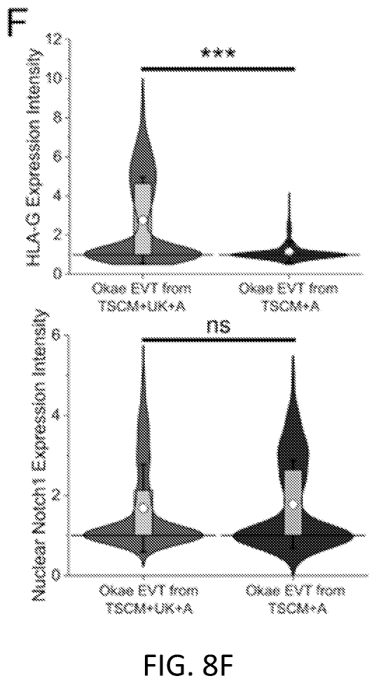

UK5099 is necessary for hTSC derivation from term CTB. Because hTSCs were effectively derived from term CTBs with the addition of UK and AlbuMAX, experiments were conducted to tease apart why these two additional components allowed for hTSC formation. First, experiments were conducted to see if UK was necessary for the derivation of hTSCs. Isolated CTBs were thawed from term placenta into TSCM+AlbuMAX without UK5099. It was found that hTSCs expressed relevant CTB markers, similar to hTSCs cultured in TSCM+UK+A including, GATA3, TEAD4, p63, CDX2, KRT7, AP-2γ, and YAP (FIG. 8A). However, AP-2γ and KRT7 expression was lower than hTSCs cultured with UK5099 and YAP staining was less nuclear (FIG. 8A). hTSCs also did not express OCT4, nor HIF1α, and had low HIF2α expression, as did the hTSCs cultured with UK (FIG. 8A). However, when quantitative analysis was performed, it was found that while hTSCs cultured without UK had comparable nuclear p63 expression, yet nuclear CDX2 expression was significantly lower (FIG. 8B). As previously mentioned, hTSCs lost CDX2 expression with the removal of AlbuMAX and UK5099 (FIG. 8C). However, CDX2 expression was seen when cells were thawed in TSCM alone (FIG. 7C). Experiments were then conducted to differentiate these cells cultured in TSCM+AlbuMAX and found that neither differentiation protocols led to EVT or STB. Cells obtained from EVT differentiation using the 1-Step protocol were Notch1+ but expression of Notch1 was significantly lower than cells cultured with UK and these cells did not express HLA-G (FIGS. 8C and 8D). Additionally, STB did not express high levels of hCG nor SDC-1 (FIG. 8C). Cells obtained from EVT differentiation using the Okae protocol were negative for HLA-G and had comparable Notch1 expression to hTSCs derived from TSCM+UK+A (FIGS. 8E and 8F). Cells obtained from STB differentiation using the Okae protocol were negative for both hCG and SDC-1 (FIG. 8E).

### Example 9

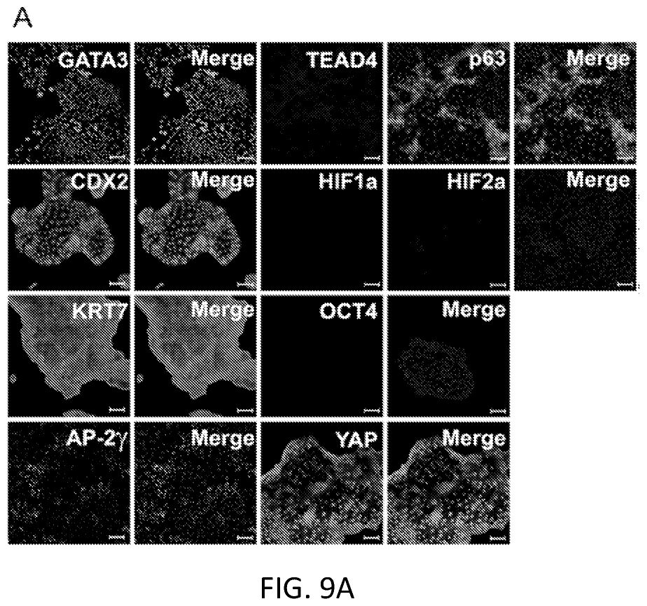

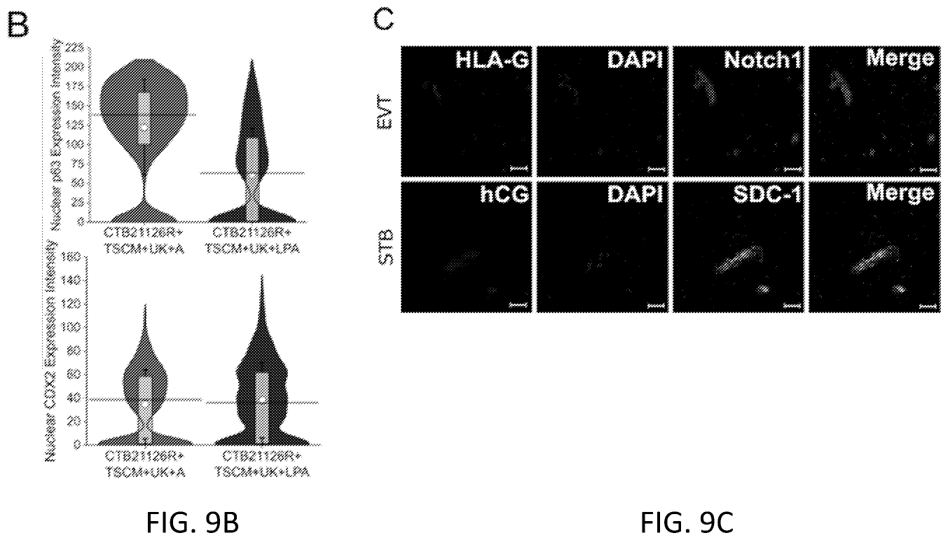

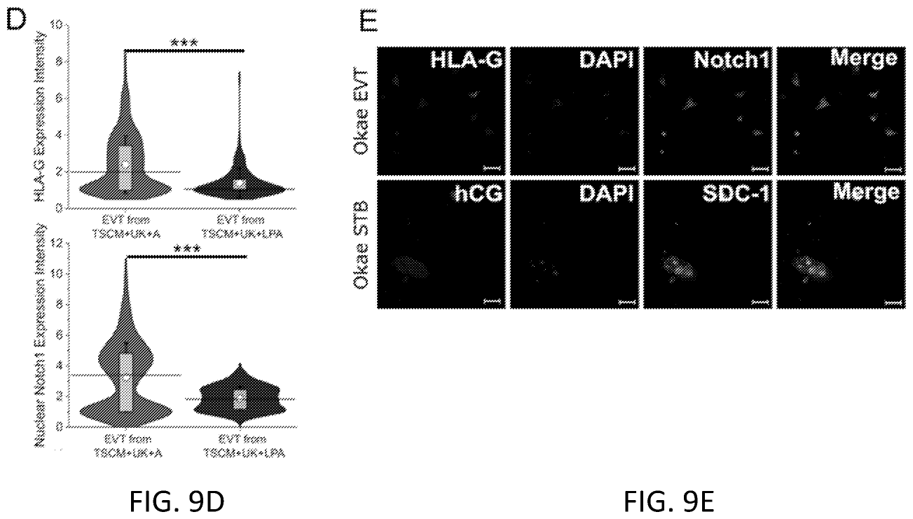

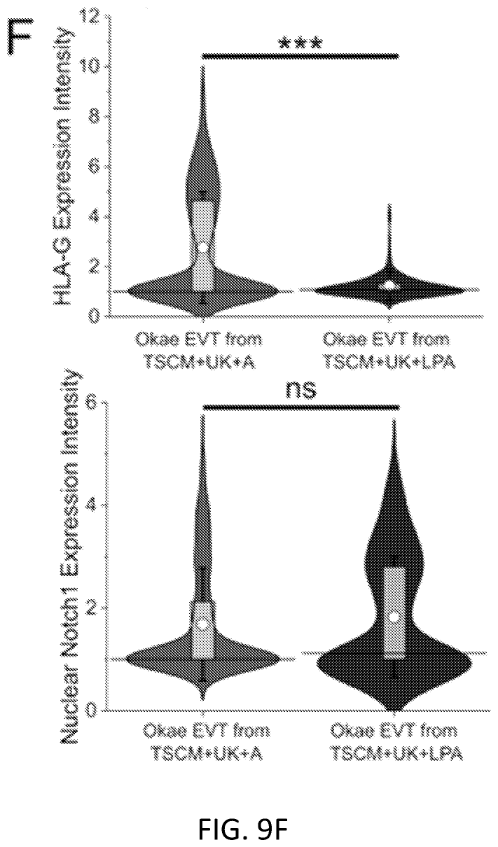

LPA cannot replace AlbuMAX in hTSC derivation from term CTB. Having the knowledge that UK5099 was necessary for the formation of hTSCs, experiments were conducted to determine whether substituting lysophosphatidic acid (LPA) with AlbuMAX would suffice in generating hTSCs. Term CTBs were thawed into TSCM+UK+LPA. Again, it was found that hTSCs expressed relevant CTB markers, similar to hTSCs cultured in TSCM+UK+A and TSCM+A. These markers include GATA3, CDX2, KRT7, and not HIF1α nor OCT4 (FIG. 9A). However, similar to TSCM+A cells and unlike TSCM+UK+A hTSCs, these cells expressed low levels of AP-2γ and had more non-nuclear YAP staining (FIG. 9A). Additionally, these cells expressed significantly lower levels of TEAD4, HIF2α, and had more non-nuclear p63 staining than the other two conditions (FIG. 9A). Quantitative analysis confirmed this; nuclear CDX2 expression was comparable with TSCM+UK+A but nuclear p63 expression was much lower (FIG. 9B). These cells were unable to differentiate to EVT or STB using the 1-Step or the Okae protocols, similar to cells cultured in TSCM+AlbuMAX (FIGS. 9C-9F).

What is claimed is:

1. A chemically defined cell culture medium for inducing and maintaining human trophoblast stem cells (hTSCs) from cytotrophoblasts (CTBs), the medium comprising: a GSK3β inhibitor; an activin/nodal inhibitor; and at least one growth factor.

2. The medium of claim 1, wherein the CTBs are obtained from a human placenta at birth.

3. The medium of claim 1, wherein the CTBs are obtained during or after the second trimester of human pregnancy.

4. The medium of any one of claims 1 to 3, wherein the hTSCs exhibit altered expression of one or more of CDX2, TFAP2C, YAP, TEAD4, KRT7, p63, and GATA3.

5. The medium of any one of claims 1 to 3, wherein the hTSCs express CDX2.

6. The medium of any one of claims 1 to 5, wherein the medium further comprises nicotinamide, nicotinamide riboside, nicotinamide mononucleotide or derivatives, variants, and salts thereof.

7. The medium of claim 6, wherein the nicotinamide is present in the medium at a concentration from about 1 mM to about 20 mM.

8. The medium of any one of claims 1 to 7, wherein the medium further comprises lactate, or derivatives, variants, and salts thereof.

9. The medium of claim 8, wherein the lactate is sodium lactate and is present in the medium at a concentration from about 2 mM to about 20 mM.

10. The medium of any one of claims 1 to 9, wherein the at least one growth factor is fibroblast growth factor 10 (FGF10), hepatocyte growth factor (HGF), and/or epidermal growth factor (EGF), and any derivatives or variants thereof.

11. The medium of any one of claims 1 to 10, wherein the at least one growth factor is present in the media at a concentration from about 1 ng/mL to about 100 ng/mL.

12. The medium of any one of claims 1 to 11, wherein the medium further comprises a sphingosine 1-phosphate receptor (S1PR) agonist.

13. The medium of claim 12, wherein the S1PR agonist is an agonist of S1PR1, S1PR2, or S1PR3.

14. The medium of claim 12, wherein the S1PR agonist is S1P.

15. The medium of any one of claims 12 to 14, wherein the S1PR agonist is selected from the group consisting of CYM5442, CYM5541, CYM5520, A971432, Ceralifimod, CS2100, CYM50260, CYM50308, FTY720, GSK2018682, RP001, SEW2871, TC-G1006, TC-SP14, and any derivatives or variants thereof.

16. The medium of any one of claims 12 to 14, wherein the S1PR agonist is an agonist of S1PR2.

17. The medium of any one of claims 12 to 16, wherein the S1PR agonist is present in the medium at a concentration from about 1 μM to about 10 μM.

18. The medium of any one of claims 1 to 17, wherein the GSK3β inhibitor is CHIR99021 or any derivatives or variants thereof.

19. The medium of any one of claims 1 to 18, wherein the GSK3β inhibitor is present in the medium at a concentration from about 0.5 μM to about 5 μM.

20. The medium of any one of claims 1 to 19, wherein the activin/nodal inhibitor is SB431542 or A83-01, and any derivatives or variants thereof.

21. The medium of any one of claims 1 to 20, wherein the activin/nodal inhibitor is present in the medium at a concentration from about 0.2 μM to about 4 μM.

22. The medium of any one of claims 1 to 21, wherein the medium comprises ascorbic acid at a concentration from about 25 μg/mL to about 150 μg/mL.

23. The medium of any one of claims 1 to 22, wherein the medium further comprises lysophosphatidic acid (LPA), and/or an LPA receptor agonist.

24. The medium of claim 23, wherein the LPA receptor agonist comprises 2-[[3-(1,3-dioxo-1H-benz[de]isoquinolin-2(3H)-yl)propyl]thio]benzoic acid (GRI 977143) or 1-O-9Z-Octadecenoyl-sn-glyceryl-3-phosphoric acid (1-Oleoyl lysophosphatidic acid), or any salts thereof.

25. The medium of any one of claims 1 to 24, wherein the medium further comprises decanoic acid and/or dimethyl alpha-ketoglutarate (DMKG).

26. The medium of any one of claims 1 to 25, wherein the medium comprises DMEM and/or F12 basal medium.

27. The medium of any one of claims 1 to 26, wherein the medium further comprises glucose at a concentration of 20 mM or less.

28. The medium of any one of claims 1 to 27, wherein the medium further comprises an inhibitor of mitochondrial pyruvate uptake.

29. The medium of claim 28, wherein the mitochondrial pyruvate uptake inhibitor comprises α-cyano-β-(1-phenylindol-3-yl)-acrylate (UK5099).

30. The medium of claim 28, wherein the mitochondrial pyruvate uptake inhibitor is present in the medium at a concentration ranging from about 1 nM to about 500 nM.

31. The medium of any one of claims 1 to 30, wherein the medium further comprises bovine serum albumin (BSA).

32. The medium of claim 31, wherein the BSA is a lipid-rich BSA composition.

33. The medium of claim 32, wherein the lipid-rich BSA composition comprises phospholipids and/or other hydrophobic lipids.

34. The medium of claim 33, wherein the phospholipids comprise sphingoine-1-phosphate (S1P) and/or lysophosphatidic acid (LPA).

35. The medium of claim 33, wherein the lipid-rich BSA composition is Albumax, and wherein the Albumax is present in the medium at a concentration ranging from 0.05% to about 1.0%.

36. The medium of any one of claims 1 to 35, wherein the medium comprises oxygen levels that are at least 1%.

37. A chemically defined cell culture medium for inducing and maintaining human trophoblast stem cells (hTSCs) from cytotrophoblasts (CTBs), the medium comprising: a GSK3β inhibitor; an activin/nodal inhibitor; at least one growth factor; a histone deacetylase (HDAC) inhibitor; and a Rho-associated, coiled-coil containing protein kinase (ROCK) inhibitor.

38. The medium of claim 37, wherein the CTBs are obtained from a human placenta at birth.

39. The medium of claim 37, wherein the CTBs are obtained during or after the second trimester of human pregnancy.

40. The medium of any one of claims 37 to 39, wherein the hTSCs exhibit altered expression of one or more of CDX2, TFAP2C, YAP, TEAD4, KRT7, p63, and GATA3.

41. The medium of any one of claims 37 to 39, wherein the hTSCs express CDX2.

42. The medium of any one of claims 37 to 41, wherein the at least one growth factor is epidermal growth factor (EGF).

43. The medium of any one of claims 37 to 42, wherein the GSK3β inhibitor is CHIR99021 or any derivatives or variants thereof.

44. The medium of any one of claims 37 to 43, wherein the activin/nodal inhibitor is SB431542 or A83-01, and any derivatives or variants thereof.

45. The medium of any one of claims 37 to 44, wherein the HDAC inhibitor is valproic acid (VPA).

46. The medium of any one of claims 37 to 45, wherein the ROCK inhibitor is Y27632.

47. The medium of any one of claims 37 to 46, wherein the medium further comprises an inhibitor of mitochondrial pyruvate uptake.

48. The medium of claim 47, wherein the mitochondrial pyruvate uptake inhibitor comprises α-cyano-β-(1-phenylindol-3-yl)-acrylate (UK5099).

49. The medium of claim 47, wherein the mitochondrial pyruvate uptake inhibitor is present in the medium at a concentration ranging from about 1 nM to about 500 nM.

50. The medium of any one of claims 37 to 49, wherein the medium further comprises bovine serum albumin (BSA).

51. The medium of claim 50, wherein the BSA is a lipid-rich BSA composition.

52. The medium of claim 51, wherein the lipid-rich BSA composition comprises phospholipids and/or other hydrophobic lipids.

53. The medium of claim 52, wherein the phospholipids comprise sphingoine-1-phosphate (S1P) and/or lysophosphatidic acid (LPA).

54. The medium of claim 51, wherein the lipid-rich BSA composition is Albumax, and wherein the Albumax is present in the medium at a concentration ranging from 0.05% to about 1.0%.

55. The medium of any one of claims 37 to 54, wherein the medium comprises oxygen levels that are at least 1%.

56. A method for inducing and maintaining human trophoblast stem cells (hTSCs) from cytotrophoblasts (CTBs) ex vivo, the method comprising obtaining CTBs from placentas at birth, and culturing the CTBs in the medium of any one of claims 1 to 55 for at least 2 passages, wherein the hTSCs express CDX2.

57. An ex vivo human trophoblast stem cell (hTSC) derived from a cytotrophoblast (CTB) obtained from a placenta at birth, wherein the hTSC expresses CDX2.

58. A container comprising an ex vivo human trophoblast stem cell (hTSC) derived from a cytotrophoblast (CTB) obtained from a placenta at birth, wherein the hTSC expresses CDX2.

59. The container of claim 58, further comprising the medium of any one of claims 1 to 55.
